(19) United States
(12) Patent Application Publication
(10) Pub. No.: US 2026/0232891 A1
(43) Pub. Date: Aug. 13, 2026

(54) Conduit Device For Negative Pressure Wound Therapy System

(71) Applicant: Solventum Intellectual Properties Company, MAPLEWOOD, MN (US)

(72) Inventor: Benjamin A. PRATT, Poole (GB)

(21) Appl. No.: 19/157,263

(22) PCT Filed: Feb. 13, 2024
(86) PCT No.: PCT/IB2024/051333

§ 371 Date: Aug. 15, 2025

Related U.S. Application Data
(60) Provisional application No. 63/447,481, filed on Feb. 22, 2023.

(51) Int. Cl. A61M 1/00; A61F 13/0203; A61F 13/05

(52) U.S. Cl. CPC A61M 1/912; A61F 13/022; A61F 13/05

(57) ABSTRACT

A conduit device for use with a negative pressure wound therapy system includes a tubular structure configured to fluidly connect to each of a therapy device and a wound dressing located at a wound site. The tubular structure includes a first film, a second film, and a third film at least partially disposed between and connected to each of the first film and the second film. The tubular structure includes a plurality of filters spaced apart from each other, connected to the third film, to provide pneumatic communication between a first chamber and a second chamber of the tubular structure. Each of the plurality of filters is offset from the wound port. The tubular structure includes an absorbent disposed in at least one of the first chamber and the second chamber and offset from the wound port.

### CROSS-REFERENCE TO RELATED APPLICATIONS

This application is a U.S. National Stage Entry of PCT International Application No. PCT/IB2024/051333, filed Feb. 13, 2024, which claims the benefit of priority to U.S. Provisional Application No. 63/447,481, filed on Feb. 22, 2023, which is incorporated herein by reference in its entirety.

### TECHNICAL FIELD

The present disclosure generally relates to a negative pressure wound therapy (NPWT) system and more particularly to a conduit device for use with the NPWT system.

### BACKGROUND

Caring for wounds is important in a healing process. Wounds generally produce fluids, commonly referred to as exudate. The exudate may include slough, necrotic tissue, or microbial load (e.g., bacteria and biofilms). If not properly addressed, the exudate at a wound site can lead to infection or maceration.

Negative pressure wound therapy (NPWT) systems are embodied as sealed wound-care systems particularly indicated for wounds, such as, chronic persistent wounds and/or complicated wounds. Specifically, for promoting wound healing, a pressure that is reduced relative to the surroundings (commonly referred to as “negative pressure”) is applied to the wound site. The negative pressure causes mechanical contraction of the wound and removal of the exudate from the wound site, thus promoting formation of granulation tissues and accelerating wound healing. The NPWT system typically includes a therapy unit that is in fluid communication with the wound site.

The exudate removed from the wound site is generally collected in a rigid canister for disposal or analysis. Typically, to manage low levels of the exudate, the use of rigid canisters for storing the exudate may increase patient discomfort as they may make the NPWT system bulky to handle and may affect a mobility of patient. Further, such rigid canisters may also impact a portability of the NPWT system.

In some examples, conventional wound dressings or bridge dressings may include a superabsorbent to absorb low levels of exudates. The superabsorbent is typically disposed at a wound interface such that the superabsorbent may directly communicate with the wound site. When such wound dressings or bridge dressings get saturated with the exudate, the wound dressings or bridge dressings may adversely affect a transfer of the negative pressure to the wound site as the exudate may block pathways for negative pressure flow, which may lead to loss of therapy. In other cases, the exudate collected in the wound dressing or bridge dressing itself may present a risk of skin maceration if the absorbent is fully saturated. Further, some wound dressings or bridge dressings may not completely isolate components of the NPWT system, such as, the pump from the exudate. Thus, some wound dressings or bridge dressings may allow ingress of the exudate into the components of the NPWT system, which may contaminate and/or damage the components of the NPWT system.

### SUMMARY

Generally, the present disclosure relates to a conduit device and a negative pressure wound therapy system including the conduit device.

In a first aspect, the present disclosure provides a conduit device for use with a negative pressure wound therapy system. The conduit device includes a tubular structure configured to fluidly connect to each of a therapy device and a wound dressing located at a wound site. The tubular structure extends along a longitudinal axis and a lateral axis orthogonal to the longitudinal axis. The tubular structure defines a first end and a second end opposite the first end. The second end is disposed proximal to the wound dressing. The tubular structure includes a first film that is fluid impermeable. The tubular structure further includes a second film connected to the first film. The second film includes a wound port disposed proximal to the second end of the tubular structure and fluidly communicating with the wound dressing. The first film and the second film together form an interior of the tubular structure. The second film is fluid impermeable. The tubular structure further includes a third film at least partially disposed between and connected to each of the first film and the second film. The third film extends along the longitudinal axis and separates the interior into a first chamber fluidly communicating with the therapy device and a second chamber. The third film is fluid impermeable and fluidly separates the first chamber from the second chamber. The wound port fluidly communicates the second chamber with the wound dressing. The tubular structure further includes a plurality of filters spaced apart from each other along the longitudinal axis. Each of the plurality of filters is connected to the third film and is configured to pneumatically communicate the first chamber with the second chamber. Each of the plurality of filters is offset from the wound port with respect to at least one of the longitudinal axis and the lateral axis. The tubular structure further includes an absorbent disposed in at least one of the first chamber and the second chamber and offset from the wound port with respect to at least one of the longitudinal axis and the lateral axis.

In a second aspect, the present disclosure provides a negative pressure wound therapy system. The negative pressure wound therapy system includes a therapy device including a negative pressure source and a battery pack. The negative pressure wound therapy system further includes a wound dressing located at a wound site. The negative pressure wound therapy system further includes the conduit device of the first aspect. The wound port of the conduit device is disposed in fluid communication with the wound dressing. The first chamber is disposed in fluid communication with the therapy device.

In a third aspect, the present disclosure provides a conduit device for use with a negative pressure wound therapy system. The conduit device includes a tubular structure configured to fluidly connect to each of a therapy device and a wound dressing located at a wound site. The tubular structure extends along a longitudinal axis. The tubular structure defines a first end and a second end opposite the first end. The second end is disposed proximal to the wound dressing. The tubular structure includes a first film that is fluid impermeable. The tubular structure further includes a second film connected to the first film. The second film includes a wound port disposed proximal to the second end of the tubular structure and fluidly communicating with the wound dressing. The first film and the second film together form an interior of the tubular structure. The second film is fluid impermeable. The tubular structure further includes a third film at least partially disposed between and connected to each of the first film and the second film. The third film extends along the longitudinal axis and separates the interior into a first chamber fluidly communicating with the therapy device and a second chamber. The third film is fluid impermeable and fluidly separates the first chamber from the second chamber. The wound port fluidly communicates the second chamber with the wound dressing. The first chamber defines a first length along the longitudinal axis and the second chamber defines a second length along the longitudinal axis. The first length is equal to the second length. The tubular structure further includes a plurality of filters spaced apart from each other along the longitudinal axis. Each of the plurality of filters is connected to the third film and is configured to pneumatically communicate the first chamber with the second chamber. Each of the plurality of filters is offset from the wound port with respect to the longitudinal axis.

In a fourth aspect, the present disclosure provides a negative pressure wound therapy system. The negative pressure wound therapy system includes a therapy device including a negative pressure source and a battery pack. The negative pressure wound therapy system further includes a wound dressing located at a wound site. The negative pressure wound therapy system further includes the conduit device of the third aspect. The wound port of the conduit device is disposed in fluid communication with the wound dressing. The first chamber is disposed in fluid communication with the therapy device.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic top view of a negative pressure wound therapy (NPWT) system, according to an embodiment of the present disclosure;

FIG. 2A is a schematic perspective view of a conduit device associated with the NPWT system of FIG. 1, according to an embodiment of the present disclosure;

FIG. 2B is a schematic exploded view of the conduit device of FIG. 2A;

FIG. 2C is a schematic sectional side view of the conduit device of FIG. 2A along a line X-X1;

FIG. 3 is a schematic sectional side view of a conduit device that may be associated with the NPWT system of FIG. 1, according to another embodiment of the present disclosure;

FIG. 4 is a schematic sectional side view of a conduit device that may be associated with the NPWT system of FIG. 1, according to yet another embodiment of the present disclosure;

FIG. 5 is a schematic sectional side view of a conduit device that may be associated with the NPWT system of FIG. 1, according to an embodiment of the present disclosure;

FIG. 6 is a schematic sectional side view of a conduit device that may be associated with the NPWT system of FIG. 1, according to another embodiment of the present disclosure;

FIG. 7A is a schematic exploded view of a conduit device that may be associated with the NPWT system of FIG. 1, according to yet another embodiment of the present disclosure;

FIG. 7B is a schematic sectional side view of the conduit device of FIG. 7A;

FIG. 8A is a schematic perspective view of a conduit device associated with the NPWT system of FIG. 1, according to an embodiment of the present disclosure;

FIG. 8B is a schematic exploded view of the conduit device of FIG. 8A;

FIG. 8C is a schematic sectional side view of the conduit device of FIG. 8A along a line Y-Y1;

FIG. 9 is a schematic sectional side view of a conduit device that may be associated with the NPWT system of FIG. 1, according to another embodiment of the present disclosure;

FIG. 10 is a schematic sectional side view of a conduit device that may be associated with the NPWT system of FIG. 1, according to yet another embodiment of the present disclosure;

FIG. 11A is a schematic perspective view of a conduit device associated with the NPWT system of FIG. 1, according to an embodiment of the present disclosure;

FIG. 11B is a schematic exploded view of the conduit device of FIG. 11A;

FIG. 11C is a schematic sectional side view of the conduit device of FIG. 11A along a line Z-Z1;

FIG. 12 is a schematic sectional side view of a conduit device that may be associated with the NPWT system of FIG. 1, according to another embodiment of the present disclosure;

FIG. 13A is a schematic sectional side view of a conduit device that may be associated with the NPWT system of FIG. 1, according to yet another embodiment of the present disclosure; and

FIG. 13B is a schematic top view of the conduit device of FIG. 13A.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As used herein, all numbers should be considered modified by the term “about”. As used herein, “a,” “an,” “the,” “at least one,” and “one or more” are used interchangeably.

The term “about”, unless otherwise specifically defined, means to a high degree of approximation (e.g., within +/−5% for quantifiable properties) but again without requiring absolute precision or a perfect match.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

The term “coupled”, or “connected” may include direct physical connections between two or more components, or indirect physical connections between two or more components that are connected together by one or more additional components. For example, a first component may be coupled to a second component by being directly connected together or by being connected by a third component.

As used herein, the term “configured to” and like is at least as restrictive as the term “adapted to” and requires actual design intention to perform the specified function rather than mere physical capability of performing such a function.

As used herein, the terms “layer,” “sheet,” and “dressing,” or variations thereof, are used to describe an article having a thickness that is small relative to its length and width.

As used herein, the term “negative pressure” broadly refers to a pressure lower than a local pressure in a local environment outside of a sealed treatment environment provided by a dressing. In many cases, the local ambient pressure can also be the atmospheric pressure at which a wound site is located. Alternatively, the pressure can be less than a hydrostatic pressure associated with a tissue at the wound site.

As used herein, the term “wounds” may include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or a defect but may be desired to add or promote additional tissue growth. For example, negative pressure therapy can be used in a particular tissue area to grow additional tissue that can be harvested or transplanted to another tissue site. The wound site may also include an area wherein a surgical incision has been previously performed.

As used herein, the terms “pneumatically communicate” and “pneumatic communication” imply, in part, that gas or gas pressure may be in communication between designated components or locations. For example, when two components pneumatically communicate with each other, a reduced/negative pressure may flow from one component to another, while restricting any passage of liquids, such as, a wound exudate, therethrough. Similarly, when a component is said to provide pneumatic communication, the component may allow passage of the reduced/negative pressure therethrough while restricting any passage of liquids, such as, the wound exudate.

As used herein, the term “fluid impermeable” may mean that a material or a component may prevent passage of fluids, including liquids and gases, therethrough.

As used herein, the term “interior” of a tubular structure may refer to an internal volume or space defined by the tubular structure.

Negative pressure wound therapy (NPWT) systems are often used to promote wound healing. In order to heal a wound, a negative pressure is applied at a wound site via the NPWT system. Since the NPWT system fluidly communicates with the wound site, the NPWT system removes a fluid, i.e., a wound exudate from the wound site by applying the negative pressure on a wound dressing attached to the wound site. The wound exudate may include slough, necrotic tissue, microbial load (e.g., bacteria and biofilms), and the like. Further, the wound exudate is collected in a canister for disposal or analysis. The canister of the NPWT system generally includes a rigid canister. Typically, to manage low levels of the wound exudate, the use of rigid canisters may increase patient discomfort as they may make the NPWT system bulky to handle and may affect a mobility of patient. Further, such rigid canisters may also impact a portability of the NPWT system. Thus, rigid canisters may be bulky, and may confine a patient to bed or at least render the patient immobile and may lead to patient discomfort.

In some examples, conventional wound dressings or bridge dressings may include a superabsorbent to absorb low levels of the wound exudate. The superabsorbent is typically disposed at a wound interface such that the superabsorbent may directly communicate with the wound site. When such wound dressings or bridge dressings get saturated with the wound exudate, the wound dressings or bridge dressings may adversely affect a transfer of the negative pressure to the wound site as the wound exudate may block pathways for negative pressure flow, which may lead to loss of therapy. In other cases, the wound exudate collected in the wound dressing or bridge dressing itself may present a risk of skin maceration if the absorbent is fully saturated. Further, some wound dressings or bridge dressings may not completely isolate components of the NPWT system, such as, the pump from the wound exudate. Thus, some wound dressings or bridge dressings may allow ingress of the wound exudate into the components of the NPWT system, which is not desirable.

Therefore, there exists a need for a device that, when used with an NPWT system, may allow collection of the wound exudate from the wound without disrupting negative pressure therapy at the wound site, without causing skin maceration, and without impacting patient comfort, while improving an ease of use, a portability, and an efficacy of the NPWT system.

The present disclosure relates to a conduit device for use with a negative pressure wound therapy system. The conduit device includes a tubular structure configured to fluidly connect to each of a therapy device and a wound dressing located at a wound site. The tubular structure extends along a longitudinal axis and a lateral axis orthogonal to the longitudinal axis. The tubular structure defines a first end and a second end opposite the first end. The second end is disposed proximal to the wound dressing. The tubular structure includes a first film that is fluid impermeable. The tubular structure further includes a second film connected to the first film. The second film includes a wound port disposed proximal to the second end of the tubular structure and fluidly communicating with the wound dressing. The first film and the second film together form an interior of the tubular structure. The second film is fluid impermeable. The tubular structure further includes a third film at least partially disposed between and connected to each of the first film and the second film. The third film extends along the longitudinal axis and separates the interior into a first chamber fluidly communicating with the therapy device and a second chamber. The third film is fluid impermeable and fluidly separates the first chamber from the second chamber. The wound port fluidly communicates the second chamber with the wound dressing. The tubular structure further includes a plurality of filters spaced apart from each other along the longitudinal axis. Each of the plurality of filters is connected to the third film and is configured to pneumatically communicate the first chamber with the second chamber. Each of the plurality of filters is offset from the wound port with respect to at least one of the longitudinal axis and the lateral axis. The tubular structure further includes an absorbent disposed in at least one of the first chamber and the second chamber and offset from the wound port with respect to at least one of the longitudinal axis and the lateral axis.

The conduit device provides an effective way of managing low levels of the wound exudate removed from the wound site. In one particular embodiment, the conduit device may provide a multi-directional flexible canister which can be used in a range of different form factors or orientations as a multi-point pressure manifold solution. Specifically, the conduit device may provide the multi-point pressure manifold solution by providing the first and second chambers with a plurality of fluid transfer points, such as the filters, that permit pneumatic communication between the two chambers. One of the two chambers can store fluids and the wound exudate from the wound site, and another of the two chambers can bridge a port fluidly coupled to the therapy device. Further, multiple orientation use of the conduit device may permit use of the conduit device in a wider variety of locations and persons. In addition, multiple orientation use of the conduit device may permit the conduit device to be used in a mobile environment. Therefore, the conduit device may eliminate the requirement of a separate rigid canister to store the wound exudate thereby reducing a weight of the NPWT system, while improving a portability of the NPWT system.

The offset between the absorbent and the wound port may prevent any contact of the wound exudate absorbed by the absorbent and the wound site. Further, the offset between the plurality of filters and the wound port may substantially reduce any direct contact between the wound exudate and the plurality of filters. The at least partial alignment between the plurality of filters and the absorbent may enable the absorbent to first absorb at least a portion of the wound exudate before the wound exudate can contact the plurality of filters. This may substantially reduce clogging of the plurality of filters, thereby increasing an operational life of the plurality of filters.

Further, in some embodiments, the first film, the second film, and/or the third film may include projections thereon. In such embodiments, the projections may define a negative pressure pathway even when the absorbent is saturated with the wound exudate. Thus, the conduit device may allow effective management of the wound exudate in all orientations and may reduce a possibility of blocking of negative pressure pathways when the absorbent is saturated with the wound exudate.

Further, the absorbent is present in the conduit device instead of the wound dressing. As the absorbent is not localized, or in other words, directly interfacing the wound site, a possibility of skin maceration when the absorbent is saturated with the wound exudate may be reduced. Moreover, the third film and the filters may allow retention of the wound exudate within the second chamber and prevent passage of any wound exudate to the first chamber or towards the therapy device, thereby reducing a possibility of ingress of the wound exudate into the therapy device. Accordingly, a contamination of the components of the therapy device, such as, the negative pressure source may be prevented. Further, the conduit device may eliminate a need of hydrophobic filters between the conduit device and the negative pressure source for protecting the negative pressure source from any contamination due to contact with the wound exudate.

Overall, the conduit device as described herein may be usable in multiple orientations, may be easy to use, may improve patient comfort, may reduce a possibility of infection at the wound site, may improve an efficacy and a portability of the NPWT system, and may effectively manage the wound exudate removed from the wound site.

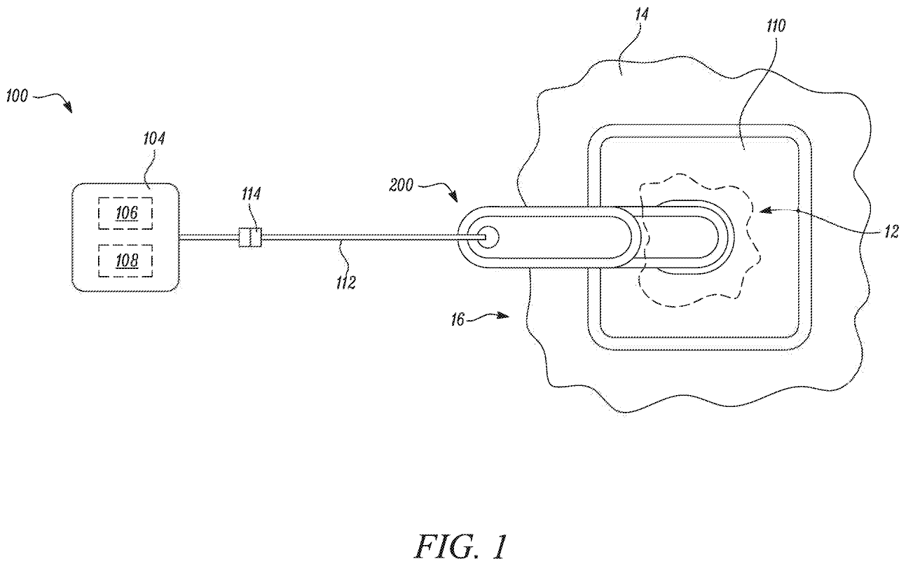

FIG. 1 is a schematic view illustrating a NPWT system 100 for treatment of a wound site 12, according to an embodiment of the present disclosure. The NPWT system 100 may be disposed on a skin 14 of a user 16. The user 16 is a patient having a wound (not shown). The skin 14 of the user 16 includes the wound site 12. The NPWT system 100 includes a therapy device 104 including a negative pressure source 106 and a battery pack 108. In some examples, the negative pressure source 106 may include a reservoir of air at a negative pressure or a manual or electrically powered device, such as, a pump. The pump may include, for example, a vacuum pump, a suction pump, or a micro-pump.

Further, the battery pack 108 may be configured to provide power to the negative pressure source 106. In some examples, the battery pack 108 may include one or more batteries including, but not limited to, a nickel cadmium battery, a nickel metal hydride battery, a lithium-ion battery, a small-sealed lead acid battery, an alkaline battery, or other suitable type of battery. The battery pack 108 may be removable, may be wirelessly chargeable, and the like.

The NPWT system 100 further includes a wound dressing 110 located at the wound site 12. The wound dressing 110 faces the wound site 12 and is in direct fluid communication with the wound site 12. The NPWT system 100 further includes a conduit device 200. The conduit device 200 is coupled to the therapy device 104 via a tubing 112 and a connector 114. In some examples, the connector 114 may include a pressure regulator for regulating a pressure level at the wound site 12.

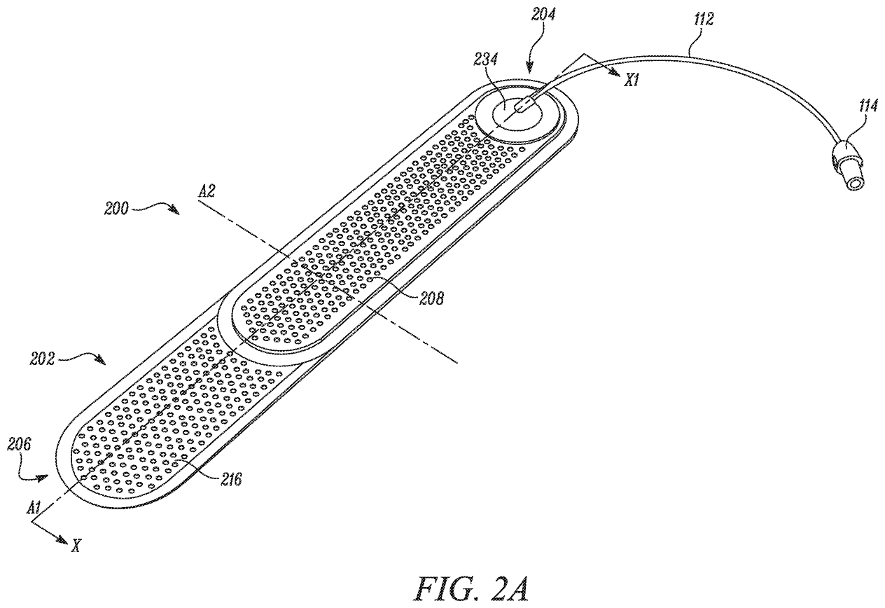

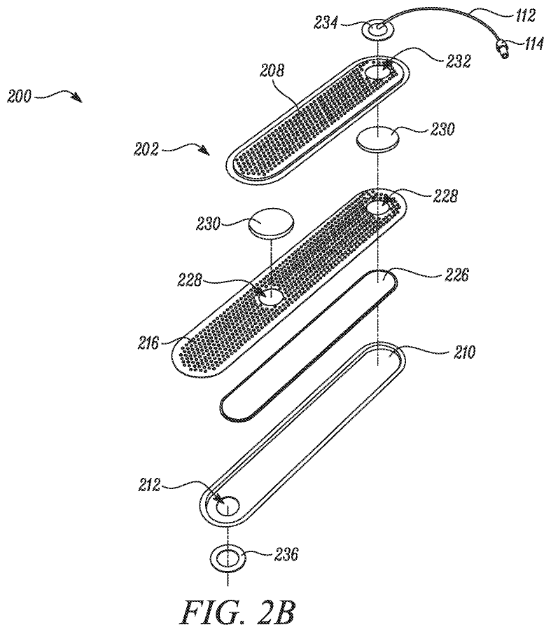

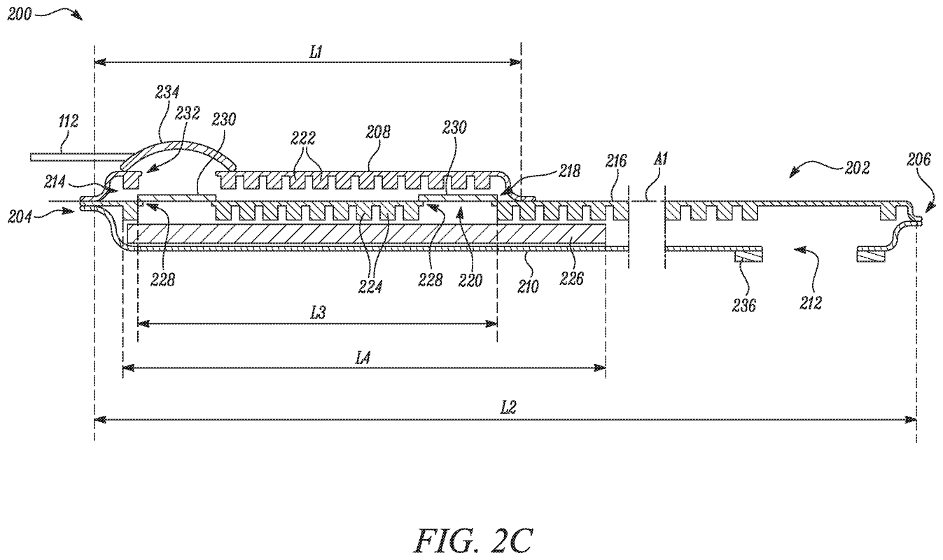

FIGS. 2A and 2B illustrate the conduit device 200 for use with the NPWT system 100 of FIG. 1, according to an embodiment of the present disclosure. The conduit device 200 includes a tubular structure 202 configured to fluidly connect to each of the therapy device 104 (see FIG. 1) and the wound dressing 110 (see FIG. 1) located at the wound site 12 (see FIG. 1). The tubular structure 202 extends along a longitudinal axis A1 and a lateral axis A2 orthogonal to the longitudinal axis A1. Further, the tubular structure 202 defines a first end 204 and a second end 206 opposite the first end 204. The second end 206 is disposed proximal to the wound dressing 110. The tubular structure 202 includes a first film 208 that is fluid impermeable. The first film 208 is coupled to the tubing 112.

Referring now to FIG. 2C, the tubular structure 202 further includes a second film 210 connected to the first film 208. The second film 210 includes a wound port 212 disposed proximal to the second end 206 of the tubular structure 202 and fluidly communicating with the wound dressing 110 (see FIG. 1). The first film 208 and the second film 210 together form an interior 214 of the tubular structure 202. The second film 210 is fluid impermeable.

The tubular structure 202 further includes a third film 216 at least partially disposed between and connected to each of the first film 208 and the second film 210. The third film 216 extends along the longitudinal axis A1 and separates the interior 214 into a first chamber 218 fluidly communicating with the therapy device 104 (see FIG. 1) and a second chamber 220. The third film 216 is fluid impermeable and fluidly separates the first chamber 218 from the second chamber 220. Further, the wound port 212 fluidly communicates the second chamber 220 with the wound dressing 110. In other words, the wound port 212 is in fluid communication with the second chamber 220 and the wound dressing 110. The first chamber 218 defines a first length L1 along the longitudinal axis A1 and the second chamber 220 defines a second length L2 along the longitudinal axis A1. In the illustrated embodiment of FIG. 2C, the second length L2 is greater than the first length L1. Alternatively, the second length L2 may be equal to the first length L1.

In the illustrated embodiment of FIG. 2C, the first film 208 includes a plurality of first projections 222 extending into the first chamber 218 orthogonal to the longitudinal axis A1 and facing the third film 216. Each of the first projections 222 has a circular cross-section herein. Alternatively, each of the first projections 222 may have a square cross-section, a rectangular cross-section, an oval cross-section, a triangular cross-section, and the like. The first projections 222 provide a manifolding geometry to the first film 208 that may improve distribution of the negative pressure within the first chamber 218. In some examples, the second film 210 may also include a plurality of projections (not shown) extending into the second chamber 220 orthogonal to the longitudinal axis A1.

Further, the third film 216 includes a plurality of second projections 224 extending into the second chamber 220 orthogonal to the longitudinal axis A1 and configured to at least partially engage with an absorbent 226. Each of the second projections 224 has a circular cross-section herein. Alternatively, each of the second projections 224 may have a square cross-section, a rectangular cross-section, an oval cross-section, a triangular cross-section, and the like. The second projections 224 provide a manifolding geometry to the third film 216 that may improve distribution of the negative pressure from the therapy device 104 within the second chamber 220. Further, the third film 216 includes a plurality of filter openings 228 spaced apart from each other along the longitudinal axis A1.

Each of the first film 208, the second film 210, and the third film 216 may prevent fluid communication, including pneumatic communication, therethrough. In an example, each of the first film 208, the second film 210, and the third film 216 may include a moisture vapor transmission resistance (MVTR) film. In an example, each of the first film 208, the second film 210, and the third film 216 may be made of an elastomeric material. Examples of elastomeric material may include, but is not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate (EVA) film, co-polyester, and silicones. An additional, specific non-limiting example of each of the first film 208, the second film 210, and the third film 216 may include a 30 μm matte polyurethane film such as the Inspire™ 2317 manufactured by Exopack™ Advanced Coatings of Matthews, N.C.

Further, each of the first film 208, the second film 210, and the third film 216 may be connected to each other at each of the first end 204 and the second end 206. In an example, each of the first film 208, the second film 210, and the third film 216 may be connected to each other by a weld seam. In alternate examples, each of the first film 208, the second film 210, and the third film 216 may be connected to each other by bonding, stitching, an adhesive, a heat seal, and the like. Each of the first film 208, the second film 210, and the third film 216 may be connected to each other, such that the tubular structure 202 has a flat configuration as shown in FIG. 2A.

Further, the tubular structure 202 includes a plurality of filters 230 spaced apart from each other along the longitudinal axis A1. Each of the plurality of filters 230 is connected to the third film 216 and is configured to pneumatically communicate the first chamber 218 with the second chamber 220. Further, each of the plurality of filters 230 is offset from the wound port 212 with respect to at least one of the longitudinal axis A1 and the lateral axis A2 (see FIG. 2A). In the illustrated embodiment of FIG. 2C, each of the plurality of filters 230 is offset from the wound port 212 with respect to the longitudinal axis A1. Therefore, each of the plurality of filters 230 is offset and does not overlap with the wound port 212 with respect to the longitudinal axis A1. The plurality of filters 230 are disposed along a first linear length L3 of the third film 216 along the longitudinal axis A1. The first linear length L3 is also offset from the wound port 212 with respect to the longitudinal axis A1. In some embodiments, the first linear length L3 is decided so as to improve a performance of the conduit device 200. Preferably, a higher value of the first linear length L3 may increase the performance of the conduit device 200. Further, each of the plurality of filter openings 228 is configured to at least partially align with a corresponding filter 230 from the plurality of filters 230 therethrough. In some examples, a diameter of each filter 230 may be greater than a diameter of the filter openings 228, such that the filters 230 cover a corresponding filter opening 228. Further, when the filters 230 are connected to the third film 216, a portion of each filter 230 may overlap with the third film 216.

In the illustrated embodiment of FIG. 2C, the tubular structure 202 includes two filters 230. However, the tubular structure 202 may include more than two filters 230 based on, for example, dimensions of the tubular structure 202. In the illustrated embodiment of FIG. 2C, the two filters 230 are disposed proximal to the first and second ends 204, 206, respectively. In some examples, an additional filter may be positioned between the filters 230. Further, the filters 230 may be made from a hydrophobic filters so that the filters 230 allow the negative pressure to flow therethrough, while preventing liquids, including a wound exudate, from flowing therethrough. The wound exudate may include slough, necrotic tissue, microbial load (e.g., bacteria and biofilms), and the like. Thus, the third film 216 and the filters 230 allow retention of the wound exudate within the second chamber 220 and prevent passage of any wound exudate to the first chamber 218 or towards the therapy device 104, thereby reducing a possibility of ingress of the wound exudate into the therapy device 104. Accordingly, a contamination of components of the therapy device 104, such as, the negative pressure source 106 (see FIG. 1) may be prevented. Further, the conduit device 200 may eliminate a need of hydrophobic filters between the conduit device 200 and the negative pressure source 106 for protecting the negative pressure source 106 from contamination due to contact with the wound exudate.

The tubular structure 202 further includes the absorbent 226 disposed in at least one of the first chamber 218 and the second chamber 220 and offset from the wound port 212 with respect to at least one of the longitudinal axis A1 and the lateral axis A2. In the illustrated embodiment of FIG. 2C, the absorbent 226 is disposed in the second chamber 220 and offset from the wound port 212 with respect to the lateral axis A2. The absorbent 226 is at least partially aligned with at least one of the plurality of filters 230 with respect to the longitudinal axis A1. In the illustrated embodiment of FIG. 2C, absorbent 226 is in alignment with each filter 230. Therefore, each filter 230 overlaps with at least a corresponding portion of the absorbent 226 with respect to the longitudinal axis A1. The absorbent 226 may be configured to absorb liquids. Specifically, the absorbent 226 absorbs the wound exudate removed from the wound site 12 (see FIG. 1). The multiple filters 230 disposed in a spaced apart configuration from each other together with the absorbent 226 may improve a performance of the conduit device 200.

In the illustrated embodiment of FIG. 2C, the absorbent 226 is directly adjacent to each of the second film 210 and the third film 216. In other words, the absorbent 226 is disposed directly between the second film 210 and the third film 216 with no intervening components or films. Further, the absorbent 226 defines a second linear length L4 along the longitudinal axis A1. In the illustrated embodiment of FIG. 2C, the second linear length L4 is greater than the first linear length L3. Therefore, the first linear length L3 fully overlaps with the second linear length L4 with respect to the longitudinal axis A1. Alternatively, the second linear length L4 may be less than the first linear length L3. Further, in the illustrated embodiment of FIG. 2C, the second length L2 of the second chamber 220 is lesser than the second linear length L4 of the absorbent 226.

In an embodiment, a material of the absorbent 226 may include BASF Luquafleece 402C. However, other materials may be used to form the absorbent 226, for example, superabsorbent polymers disposed on woven and non-woven substrates, fibrous materials, non-woven superabsorbent fiber by Technical Absorbents Limited, non-woven Texsus Absortex, and the like.

Further, in some examples, when the absorbent 226 is saturated, the second projections 224 of the third film 216 may engage with the absorbent 226. Additionally, when the absorbent 226 is saturated, the first projections 222 of the first film 208 may engage with the third film 216. This way, the first and second projections 222, 224 may define negative pressure pathways even when the absorbent 226 is saturated with the wound exudate. Thus, the conduit device 200 may allow effective management of the wound exudate in all orientations and may reduce a possibility of blocking of the negative pressure pathways when the absorbent 226 is saturated with the wound exudate. Further, as the absorbent 226 is not localized, or in other words, directly interfacing the wound site 12, a possibility of skin maceration when the absorbent 226 is saturated with the wound exudate may be reduced.

The offset between the absorbent 226 and the wound port 212 may prevent any contact of the wound exudate absorbed by the absorbent 226 and the wound site 12. Further, the offset between the plurality of filters 230 and the wound port 212 may substantially reduce any direct contact between the wound exudate and the plurality of filters 230. The at least partial alignment between the plurality of filters 230 and the absorbent 226 may enable the absorbent 226 to first absorb at least a portion of the wound exudate before the wound exudate can contact the plurality of filters 230. This may substantially reduce clogging of the plurality of filters 230, thereby increasing an operational life of the plurality of filters 230.

The absorbent 226 may be connected to the second film 210 or the third film 216. For example, the absorbent 226 may be connected to the second film 210 or the third film 216 by a weld seam, bonding, stitching, an adhesive, a heat seal, and the like. Alternatively, the absorbent 226 may be connected to each of the first film 208, the second film 210, and the third film 216 at each of the first end 204 and the second end 206 by a weld seam, bonding, stitching, an adhesive, a heat seal, and the like.

Further, the first film 208, the second film 210, the third film 216, and the absorbent 226 have a same shape (see FIG. 2B) herein. However, it may be contemplated that the first film 208, the second film 210, the third film 216, and the absorbent 226 may have different shapes. Each of the first film 208, the second film 210, the third film 216, and the absorbent 226 has a capsule shape (see FIG. 2B) herein. Alternatively, each of the first film 208, the second film 210, the third film 216, and the absorbent 226 may have any other shape, such as, a square shape, a rectangular shape, or combinations thereof.

In some embodiments, the first film 208 includes a therapy port 232 disposed proximal to the first end 204 of the tubular structure 202 and fluidly communicating the first chamber 218 with the therapy device 104. In some embodiments, the tubular structure 202 includes a pad 234 connected to the first film 208 and covering the therapy port 232. The pad 234 is configured to provide fluid communication between the therapy device 104 and the therapy port 232. Further, the pad 234 may facilitate pneumatic communication between the negative pressure source 106 and the tubular structure 202. However, the pad 234 may prevent liquids to flow towards the therapy device 104. Accordingly, the pad 234 may be formed from a fluid impermeable material.

In some examples, a material of the pad 234 may be same as the material of each of the first film 208, the second film 210, and the third film 216. For example, the pad 234 may be made of an elastomeric material. Examples of elastomeric material may include, but is not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, EVA film, co-polyester, and silicones. An additional, specific non-limiting example of the pad 234 may include a 30 μm matte polyurethane film, such as, the Inspire™ 2317 manufactured by Exopack™ Advanced Coatings of Matthews, N.C.

Further, the conduit device 200 includes an adhesive film 236 configured to removably connect the second film 210 with the wound dressing 110. The adhesive film 236 includes an annular shape herein and is concentric with the wound port 212. The adhesive film 236 may include a release liner (not shown) which may be removed for removably connecting the conduit device 200 with the wound dressing 110. In some examples, the adhesive film 236 may include a medically acceptable adhesive, such as a pressure-sensitive adhesive, that extends about a portion of, a periphery of, or about all of the adhesive film 236. In other examples, adhesive film 236 may be a double-sided drape tape, a paste, a hydrocolloid, a hydrogel, a silicone gel, an organogel, or other sealing devices or elements. In some examples, the conduit device 200 may also include one of more indicators that may provide an indication when the absorbent 226 is saturated with the wound exudate.

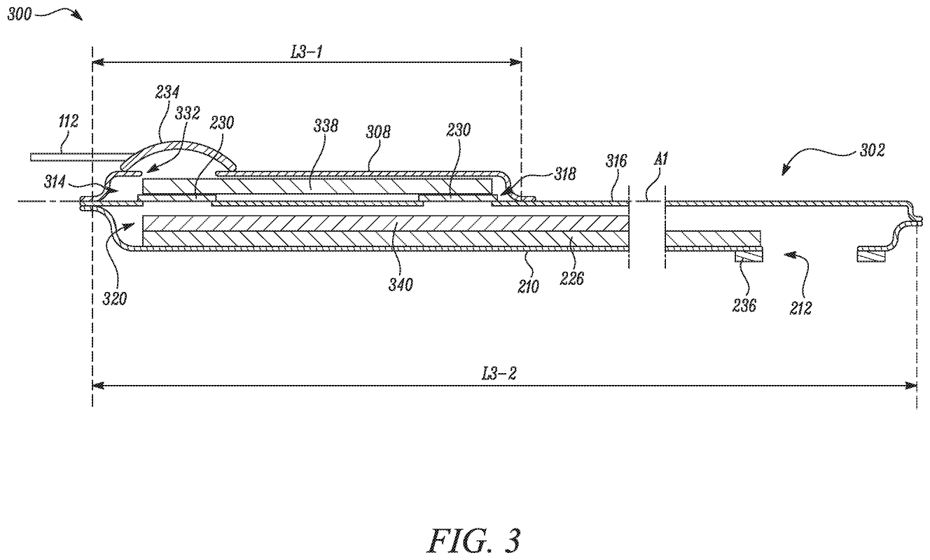

FIG. 3 is a schematic sectional side view of a conduit device 300 that may be associated with the NPWT system 100 of FIG. 1, according to another embodiment of the present disclosure. The conduit device 300 is functionally equivalent to the conduit device 200 shown in FIGS. 1 to 2C. Same parts are referred to herein by same numbers. As shown in FIG. 3, the conduit device 300 includes a tubular structure 302. The tubular structure 302 includes a first film 308 and a therapy port 332. The first film 308 is substantially similar to the first film 208 (see FIGS. 2A to 2C). However, the first film 308 does not include any projections. Further, the tubular structure 302 includes the second film 210 and the wound port 212.

The tubular structure 302 further includes a third film 316. The third film 316 is substantially similar to the third film 216 (see FIGS. 2A to 2C). However, the third film 316 does not include any projections. The third film 316 separates an interior 314 of the tubular structure 302 into a first chamber 318 fluidly communicating with the therapy device 104 (see FIG. 1) and a second chamber 320. The wound port 212 fluidly communicates the second chamber 320 with the wound dressing 110 (see FIG. 1). The first chamber 318 defines a first length L3-1 along the longitudinal axis A1 and the second chamber 320 defines a second length L3-2 along the longitudinal axis A1. In the illustrated embodiment of FIG. 3, the second length L3-2 is greater than the first length L3-1. Alternatively, the second length L3-2 may be equal to the first length L3-1.

Further, the tubular structure 302 includes the plurality of filters 230, the absorbent 226, the pad 234, and the adhesive film 236. Moreover, the tubular structure 302 includes a first manifold 338 disposed adjacent to the first film 308 within the first chamber 318. The first manifold 338 may be configured to allow passage of, or to channel, the negative pressure through the first chamber 318. In other words, the first manifold 338 may provide a flow passage across the first chamber 318. Further, the first film 308 and the first manifold 338 may be connected to each other so that the first manifold 338 may remain in position in the event that the conduit device 300 is moved, folded, or otherwise disturbed from the orientation illustrated in FIG. 3. In an example, the first film 308 may be connected to the first manifold 338 by a weld seam, bonding, stitching, an adhesive, a heat seal, and the like.

The tubular structure 302 further includes a second manifold 340 disposed adjacent to the second film 210 within the second chamber 320. The second manifold 340 is positioned between the absorbent 226 and the second film 210. The second manifold 340 may be configured to allow passage of, or to channel, the negative pressure as well as the wound exudate through the second chamber 320. In other words, the second manifold 340 may provide a flow passage across the second chamber 320. Further, in one example, the second manifold 340 may be connected to the second film 210 or the absorbent 226 so that the second manifold 340 may remain in position in the event that the conduit device 300 is moved, folded, or otherwise disturbed from the orientation illustrated in FIG. 3. In an example, the second manifold 340 may be connected to the second film 210 or the absorbent 226 by a weld seam, bonding, stitching, an adhesive, a heat seal, and the like. Alternatively, each of the first manifold 338, the second manifold 340, the first film 308, the second film 210, and the third film 316 may be connected to each other by a weld seam, bonding, stitching, an adhesive, a heat seal, and the like.

In some embodiments, each of the first and second manifolds 338, 340 may be formed from Libeltex TDL2 having a material weight of 80 grams per square member (gsm). In other embodiments, each of the first and second manifolds 338, 340 may have a material weight between about 20 gsm and about 140 gsm. Larger material weights may be selected to increase manifolding properties and the fluid capacity of each of the first and second manifolds 338, 340. Other materials may be used to form each of the first and second manifolds 338, 340, such as, woven and non-woven materials, fibrous materials, non-woven Freudenberg M1545N or M1550, non-woven Texsus Multitex, and other similar materials.

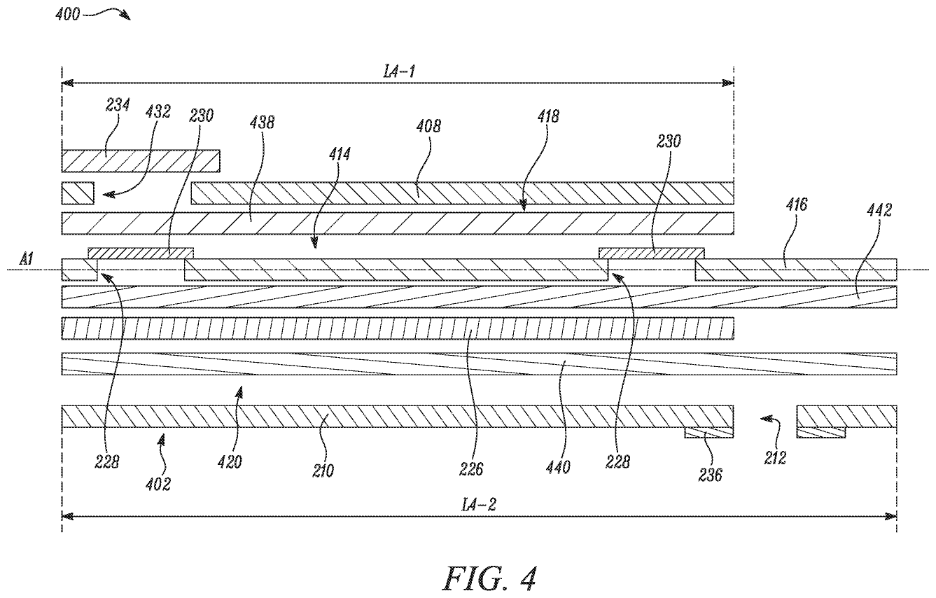

FIG. 4 is a schematic sectional side view of a conduit device 400 that may be associated with the NPWT system 100 of FIG. 1, according to yet another embodiment of the present disclosure. The conduit device 400 is functionally equivalent to the conduit device 200 shown in FIGS. 1 to 2C. Same parts are referred to herein by same numbers. As shown in FIG. 4, the conduit device 400 includes a tubular structure 402. The tubular structure 402 includes a first film 408 and a therapy port 432. The first film 408 is substantially similar to the first film 208 (see FIGS. 2A to 2C). However, the first film 408 does not include any projections. Further, the tubular structure 402 includes the second film 210 and the wound port 212.

The tubular structure 402 further includes a third film 416. The third film 416 is substantially similar to the third film 216 (see FIGS. 2A to 2C). However, the third film 416 does not include any projections. The third film 416 separates an interior 414 of the tubular structure 402 into a first chamber 418 fluidly communicating with the therapy device 104 (see FIG. 1) and a second chamber 420. The wound port 212 fluidly communicates the second chamber 420 with the wound dressing 110 (see FIG. 1). The first chamber 418 defines a first length L4-1 along the longitudinal axis A1 and the second chamber 420 defines a second length L4-2 along the longitudinal axis A1. In the illustrated embodiment of FIG. 4, the second length L4-2 is greater than the first length L4-1. Alternatively, the second length L4-2 may be equal to the first length L4-1.

Further, the tubular structure 402 includes the plurality of filters 230, the absorbent 226, the pad 234, and the adhesive film 236. Moreover, the tubular structure 402 includes a first manifold 438 disposed adjacent to the first film 408 within the first chamber 418. The first manifold 438 may be configured to allow passage of, or to channel, the negative pressure through the first chamber 418. In other words, the first manifold 438 may provide a flow passage across the first chamber 418. Further, the first film 408 and the first manifold 438 may be connected to each other so that the first manifold 438 may remain in position in the event that the conduit device 400 is moved, folded, or otherwise disturbed from the orientation illustrated in FIG. 4. In an example, the first film 408 and the first manifold 438 may be connected to each other by a weld seam, bonding, stitching, an adhesive, a heat seal, and the like.

The tubular structure 402 further includes a second manifold 440 disposed adjacent to the second film 210 within the second chamber 420. The second manifold 440 is positioned between the absorbent 226 and the second film 210. The second manifold 440 may be configured to allow passage of, or to channel, the negative pressure as well as the wound exudate through the second chamber 420. In other words, the second manifold 440 may provide a flow passage across the second chamber 420. Further, in one example, the second manifold 440 may be connected to the second film 210 or the absorbent 226 so that the second manifold 440 may remain in position in the event that the conduit device 400 is moved, folded, or otherwise disturbed from the orientation illustrated in FIG. 4. In an example, the second manifold 440 may be connected to the second film 210 or the absorbent 226 by a weld seam, bonding, stitching, an adhesive, a heat seal, and the like.

Further, the tubular structure 402 includes a third manifold 442 disposed within the second chamber 420. The third manifold 442 is positioned between the third film 416 and the absorbent 226. The third manifold 442 may be configured to allow passage of, or to channel, the negative pressure as well as the wound exudate through the second chamber 420. In other words, the third manifold 442 may provide a flow passage across the second chamber 420. Further, in one example, the third manifold 442 may be connected to the third film 416 or the absorbent 226 so that the third manifold 442 may remain in position in the event that the conduit device 400 is moved, folded, or otherwise disturbed from the orientation illustrated in FIG. 4. In an example, the third manifold 442 may be connected to the third film 416 or the absorbent 226 by a weld seam, bonding, stitching, an adhesive, a heat seal, and the like. Alternatively, the first manifold 438, the second manifold 440, the third manifold 442, the first film 408, the second film 210, and the third film 416 may be connected to each other by a weld seam, bonding, stitching, an adhesive, a heat seal, and the like. Further, materials mentioned for each of the first and second manifolds 338, 340 (see FIG. 3) may be applicable to each of the first, second, and third manifolds 438, 440, 442.

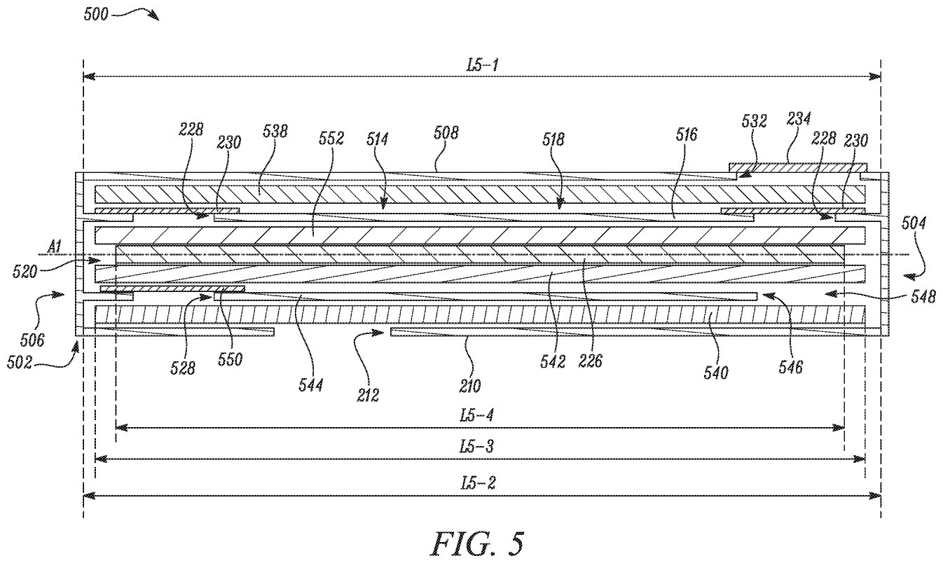

FIG. 5 is a schematic sectional side view of a conduit device 500 that may be associated with the NPWT system 100 of FIG. 1, according to an embodiment of the present disclosure. The conduit device 500 is functionally equivalent to the conduit device 200 shown in FIGS. 1 to 2C. Same parts are referred to herein by same numbers. As shown in FIG. 5, the conduit device 500 includes a tubular structure 502. The tubular structure 502 includes a first film 508 and a therapy port 532. The first film 508 is substantially similar to the first film 208 (see FIGS. 2A to 2C). However, the first film 508 does not include any projections. Further, the tubular structure 502 includes the second film 210 and the wound port 212.

The tubular structure 502 further includes a third film 516. The third film 516 is substantially similar to the third film 216 (see FIGS. 2A to 2C). However, the third film 516 does not include any projections. The third film 516 separates an interior 514 of the tubular structure 502 into a first chamber 518 fluidly communicating with the therapy device 104 (see FIG. 1) and a second chamber 520. The wound port 212 fluidly communicates the second chamber 520 with the wound dressing 110 (see FIG. 1). The first chamber 518 defines a first length L5-1 along the longitudinal axis A1 and the second chamber 520 defines a second length L5-2 along the longitudinal axis A1. In the illustrated embodiment of FIG. 5, the second length L5-2 is greater than the first length L5-1. Alternatively, the second length L5-2 may be equal to the first length L5-1.

Further, the tubular structure 502 includes the plurality of filters 230, the absorbent 226, the pad 234, and the adhesive film 236. The plurality of filters 230 are disposed along a first linear length L5-3 of the third film 216 along the longitudinal axis A1. Further, the absorbent 226 defines a second linear length L5-4 along the longitudinal axis A1. In the illustrated embodiment of FIG. 5, the second linear length L5-4 is lesser than the first linear length L5-3. Alternatively, the second linear length L5-4 may be greater than the first linear length L5-3.

Moreover, the tubular structure 502 includes a first manifold 538 disposed adjacent to the first film 508 within the first chamber 518. The first manifold 538 may be configured to allow passage of, or to channel, the negative pressure through the first chamber 518. Tin other words, the first manifold 538 may provide a flow passage across the first chamber 518. Further, the first film 508 and the first manifold 538 may be connected to each other so that the first manifold 538 may remain in position in the event that the conduit device 500 is moved, folded, or otherwise disturbed from the orientation illustrated in FIG. 5. In an example, the first film 508 and the first manifold 538 by a weld seam, bonding, stitching, an adhesive, a heat seal, and the like.

The tubular structure 502 further includes a second manifold 540 disposed adjacent to the second film 210 within the second chamber 520. The second manifold 540 may be configured to allow passage of, or to channel, the negative pressure as well as the wound exudate through the second chamber 520. In other words, the second manifold 540 may provide a flow passage across the second chamber 520. Further, in one example, the second manifold 540 may be connected to the second film 210 so that the second manifold 540 may remain in position in the event that the conduit device 500 is moved, folded, or otherwise disturbed from the orientation illustrated in FIG. 5. In an example, the second manifold 540 may be connected to the second film 210 by a weld seam, bonding, stitching, an adhesive, a heat seal, and the like.

Further, the tubular structure 502 includes a third manifold 542 disposed within the second chamber 520. The third manifold 542 is disposed adjacent to the absorbent 226. The third manifold 542 may be configured to allow passage of, or to channel, the negative pressure as well as the wound exudate through the second chamber 520. In other words, the third manifold 542 may provide a flow passage across the second chamber 520. Further, in one example, the third manifold 542 may be connected to the absorbent 226 so that the third manifold 542 may remain in position in the event that the conduit device 500 is moved, folded, or otherwise disturbed from the orientation illustrated in FIG. 5. In an example, the third manifold 542 may be connected to the absorbent 226 by a weld seam, bonding, stitching, an adhesive, a heat seal, and the like.

Further, the tubular structure 502 includes a fourth film 544 connected to each of the first film 508 and the second film 210 proximal to a first end 504 of the tubular structure 502. The tubular structure 502 further includes a second end 506 opposing the first end 504. The fourth film 544 extends along the longitudinal axis A1 and includes a free end 546 spaced apart from the second film 210 proximal to a second end 206 of the tubular structure 502, such that a fluid passage 548 is defined between the fourth film 544 and the second film 210 within the second chamber 520. The fluid passage 548 may allow the wound exudate received from the wound port 212 to flow towards the absorbent 226 for absorption. The fourth film 544 is disposed adjacent to the third manifold 542. The fourth film 544 is disposed between the second manifold 540 and the third manifold 542. Further, the fourth film 544 is fluid impermeable. In an example, the fourth film 544 may be made of an elastomeric material. Examples of elastomeric material may include, but is not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, EVA film, co-polyester, and silicones. An additional, specific non-limiting example of the fourth film 544 may include a 30 μm matte polyurethane film, such as, the Inspire™ 2317 manufactured by Exopack™ Advanced Coatings of Matthews, N.C.

Further, the tubular structure 502 includes at least one auxiliary filter 550 connected to the fourth film 544. The auxiliary filter 550 is configured to provide pneumatic communication therethrough. Further, the auxiliary filter 550 is offset from the wound port 212 with respect to the longitudinal axis A1. The auxiliary filter 550 is disposed proximal to the second end 206. In the illustrated embodiment of FIG. 5, the tubular structure 502 includes a single auxiliary filter 550. Alternatively, the tubular structure 502 may include two or more auxiliary filters. Further, a filter opening 528 in the fourth film 544 is configured to at least partially align with the auxiliary filter 550. In some examples, a diameter of each auxiliary filter 550 may be greater than a diameter of the filter opening 528, such that the auxiliary filter 550 covers the filter opening 528. Further, when the auxiliary filter 550 is connected to the fourth film 544, a portion of the auxiliary filter 550 may overlap with the fourth film 544. The auxiliary filter 550 may be embodied as a hydrophobic filter so that the auxiliary filter 550 allows the negative pressure to flow therethrough, while preventing liquids, such as the wound exudate, from flowing across the auxiliary filter 550.

Further, the tubular structure 502 includes a fourth manifold 552 disposed within the second chamber 520. The fourth manifold 552 is positioned between the third film 516 and the absorbent 226. The fourth manifold 552 may be configured to allow passage of, or to channel, the negative pressure as well as the wound exudate through the second chamber 520. In other words, the fourth manifold 552 may provide a flow passage across the second chamber 520. Further, in one example, the fourth manifold 552 may be connected to the third film 516 or the absorbent 226 so that the fourth manifold 552 may remain in position in the event that the conduit device 500 is moved, folded, or otherwise disturbed from the orientation illustrated in FIG. 5. In an example, the fourth manifold 552 may be connected to the third film 516 or the absorbent 226 by a weld seam, bonding, stitching, an adhesive, a heat seal, and the like. Alternatively, the first film 508, the second film 210, the third film 516, the first manifold 538, the second manifold 540, the third manifold 542, the fourth film 544, and the fourth manifold 552 may be connected to each other at the first end 504 by a weld seam, bonding, stitching, an adhesive, a heat seal, and the like. Moreover, the first film 508, the second film 210, the third film 516, the first manifold 538, the second manifold 540, the third manifold 542, and the fourth manifold 552 may be connected to each other at the second end 506 by a weld seam, bonding, stitching, an adhesive, a heat seal, and the like. Further, materials mentioned for each of the first and second manifolds 338, 340 (see FIG. 3) may be applicable to each of the first, second, third, and fourth manifolds 538, 540, 542, 552.

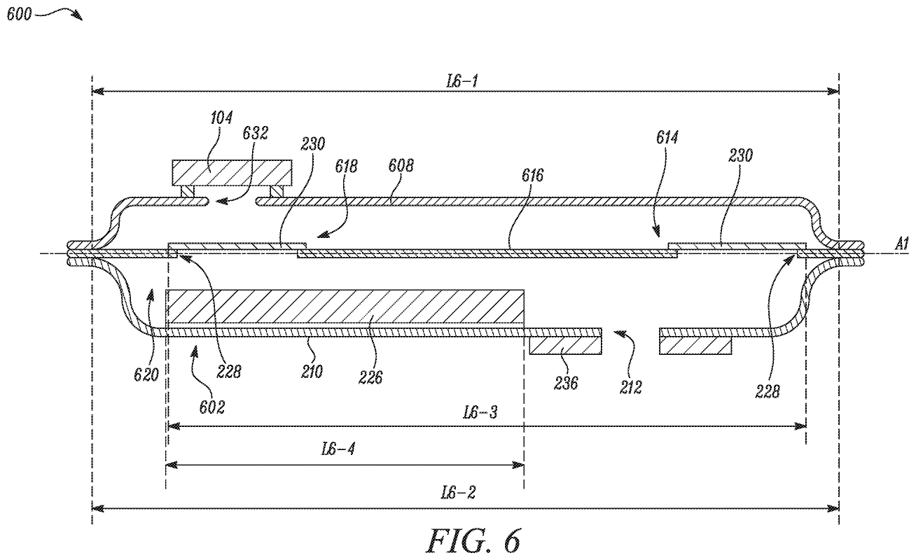

FIG. 6 is a schematic sectional side view of a conduit device 600 that may be associated with the NPWT system 100 of FIG. 1, according to another embodiment of the present disclosure. The conduit device 600 is functionally equivalent to the conduit device 200 shown in FIGS. 1 to 2C. Same parts are referred to herein by same numbers. As shown in FIG. 6, the conduit device 600 includes a tubular structure 602. The tubular structure 602 includes a first film 608 and a therapy port 632. The first film 608 is substantially similar to the first film 208 (see FIGS. 2A to 2C). However, the first film 608 does not include any projections. However, the first film 608 may include projections thereon. Further, the tubular structure 602 includes the second film 210 and the wound port 212.

The tubular structure 602 further includes a third film 616. The third film 616 is substantially similar to the third film 216 (see FIGS. 2A to 2C). However, the third film 616 does not include any projections. However, the third film 616 may include projections thereon. The third film 616 separates an interior 614 of the tubular structure 602 into a first chamber 618 fluidly communicating with the therapy device 104 (see FIG. 1) and a second chamber 620. The wound port 212 fluidly communicates the second chamber 620 with the wound dressing 110 (see FIG. 1). The first chamber 618 defines a first length L6-1 along the longitudinal axis A1 and the second chamber 620 defines a second length L6-2 along the longitudinal axis A1. In the illustrated embodiment of FIG. 6, the second length L6-2 is equal to the first length L6-1. Alternatively, the second length L6-2 may be greater than the first length L6-1.

Further, the tubular structure 602 includes the plurality of filters 230, the absorbent 226, the pad 234, and the adhesive film 236. The plurality of filters 230 are disposed along a first linear length L6-3 of the third film 216 along the longitudinal axis A1. Further, the absorbent 226 defines a second linear length L6-4 along the longitudinal axis A1. In the illustrated embodiment of FIG. 6, the second linear length L6-4 is lesser than the first linear length L6-3. Alternatively, the second linear length L6-4 may be greater than the first linear length L6-3.

Further, the NPWT system 100 includes the therapy device 104. In the illustrated embodiment of FIG. 6, the therapy device 104 is integral with the tubular structure 602 and connected to the first film 608. Specifically, the therapy device 104 is coupled to the therapy port 632 and is in direct fluid communication with the therapy port 632. In such embodiments, the conduit device 600 may eliminate a step of coupling the therapy device 104 with the tubular structure 602, thereby allowing quicker assembly of the NPWT system 100. Further, the conduit device 600 may also eliminate a requirement of the tubing 112 (see FIG. 1) and the connector 114 (see FIG. 1).

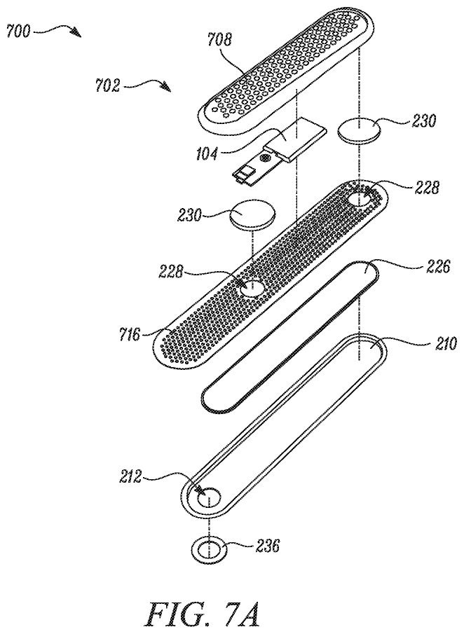

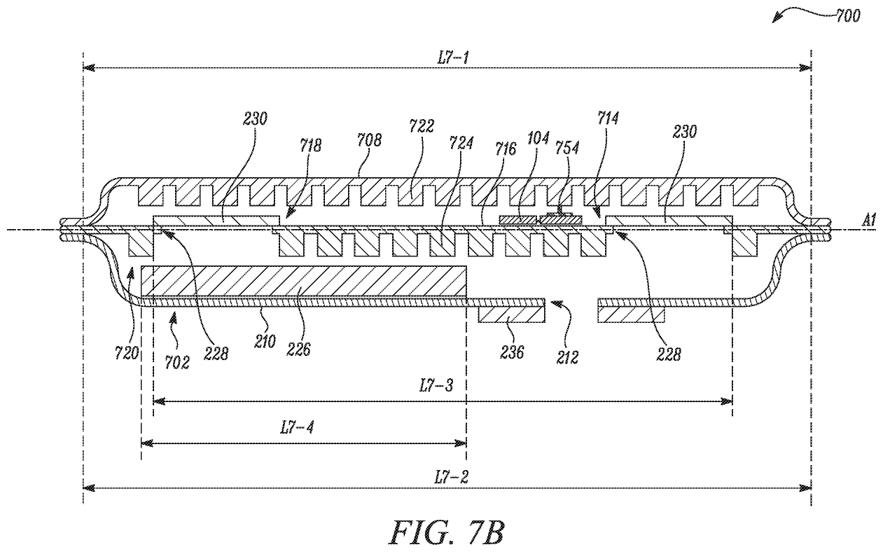

FIGS. 7A and 7B illustrate a conduit device 700 that may be associated with the NPWT system 100 of FIG. 1, according to yet another embodiment of the present disclosure. The conduit device 700 is functionally equivalent to the conduit device 200 shown in FIGS. 1 to 2C. Same parts are referred to herein by same numbers. As shown in FIGS. 7A and 7B, the conduit device 700 includes a tubular structure 702. The tubular structure 702 includes a first film 708 that is substantially similar to the first film 208 (see FIGS. 2A to 2C). The first film 708 includes a number of first projections 722. Further, the tubular structure 702 includes the second film 210 and the wound port 212.

The tubular structure 702 further includes a third film 716 having a number of second projections 724. The third film 716 is substantially similar to the third film 216 (see FIGS. 2A to 2C). Referring to FIG. 7B, the third film 716 separates an interior 714 of the tubular structure 702 into a first chamber 718 fluidly communicating with the therapy device 104 and a second chamber 720. The wound port 212 fluidly communicates the second chamber 720 with the wound dressing 110 (see FIG. 1). The first chamber 718 defines a first length L7-1 along the longitudinal axis A1 and the second chamber 720 defines a second length L7-2 along the longitudinal axis A1. In the illustrated embodiment of FIG. 7B, the second length L7-2 is equal to the first length L7-1. Alternatively, the second length L7-2 may be greater than the first length L7-1. Further, the tubular structure 702 includes the plurality of filters 230, the absorbent 226, the pad 234, and the adhesive film 236. The plurality of filters 230 are disposed along a first linear length L7-3 of the third film 216 along the longitudinal axis A1. Further, the absorbent 226 defines a second linear length L7-4 along the longitudinal axis A1. In the illustrated embodiment of FIG. 7B, the second linear length L7-4 is lesser than the first linear length L7-3. Alternatively, the second linear length L7-4 may be greater than the first linear length L7-3.

Referring to FIG. 7B, the NPWT system 100 includes the therapy device 104. However, as shown in FIG. 7B, the therapy device 104 is disposed within the first chamber 718 and adjacent to the first film 708. Specifically, the therapy device 104 is in direct fluid communication with the first chamber 718. Further, the therapy device 104 is disposed between the filters 230. In an example, a gasket 754 may connect the therapy device 104 to the first film 708. Alternatively, the gasket 754 may connect the therapy device 104 to the third film 716. In other examples, the therapy device 104 may be connected to the first film 708 or the third film 716 by bonding, an adhesive, and the like. The conduit device 700 may eliminate a step of coupling the therapy device 104 with the tubular structure 702, thereby allowing quicker assembly of the NPWT system 100. Further, the conduit device 700 may also eliminate a requirement of the tubing 112 (see FIG. 1) and the connector 114 (see FIG. 1).

Referring to FIGS. 1 to 7C, the absorbent 226 is disposed within the conduit device 200, 300, 400, 500, 600, 700 instead of the wound dressing 110. Thus, when the absorbent 226 is saturated, the conduit device 200, 300, 400, 500, 600, 700 may be replaced without disturbing the wound dressing 110 from the wound site 12. Moreover, the absorbent 226 is offset from the wound port 212 due to which the conduit device 200, 300, 400, 500, 600, 700 may reduce a possibility of skin maceration when the absorbent 226 is saturated with the wound exudate.

Further, each conduit device 200, 300, 400, 500, 600, 700 may be disposed in a variety of configuration/orientations. For example, in a first configuration, the wound port 212 of the corresponding conduit device 200, 300, 400, 500, 600, 700 may be disposed at a higher elevation as compared to the corresponding therapy port 232, 332, 432, 532, 632. In such embodiments, the conduit device 200, 300, 400, 500, 600, 700 may demonstrate a negligible to zero drop in a pressure measured at the wound port 212 and may also demonstrate improved performance.

In a second configuration, the therapy port 232, 332, 432, 532, 632 of the corresponding conduit device 200, 300, 400, 500, 600, 700 may be disposed at a higher elevation as compared to the corresponding wound port 212. In such embodiments, the conduit device 200, 300, 400, 500, 600, 700 may demonstrate a minimum or reduced drop in a pressure measured at the wound port 212 when compared to conventional bridge dressings. Further, the conduit device 200, 300, 400, 500, 600, 700 may exhibit improved performance over conventional bridge dressings.

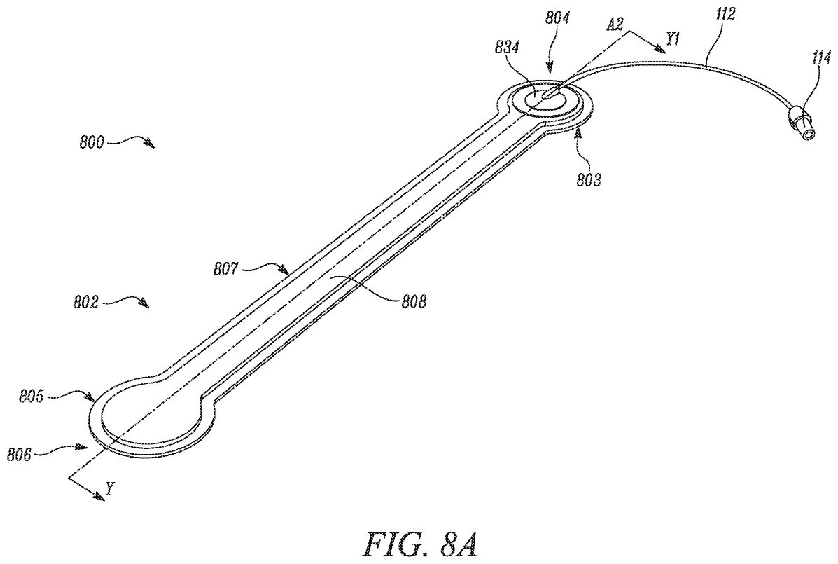

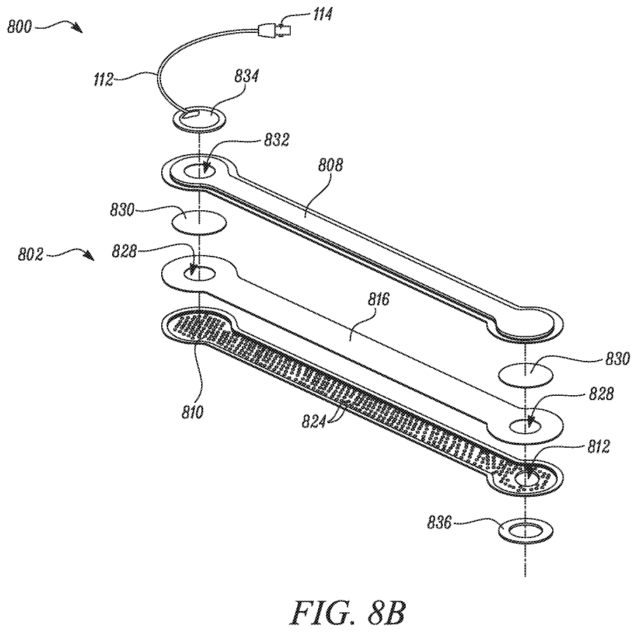

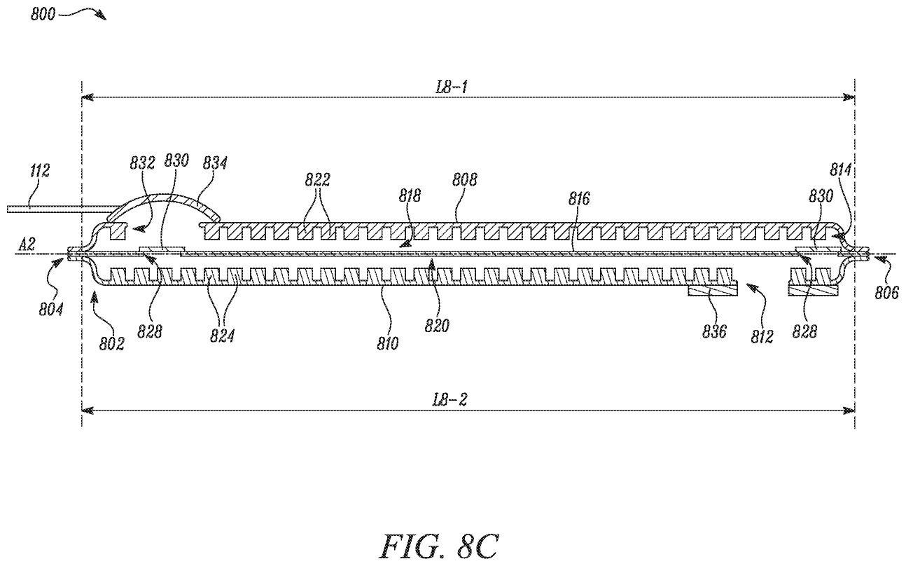

FIGS. 8A and 8B illustrate a conduit device 800 for use with the NPWT system 100 of FIG. 1, according to yet another embodiment of the present disclosure. As shown in FIGS. 8A and 8B, the conduit device 800 includes a tubular structure 802 configured to fluidly connect to each of the therapy device 104 (see FIG. 1) and the wound dressing 110 (see FIG. 1) located at the wound site 12 (see FIG. 1). The tubular structure 802 extends along a longitudinal axis A2. Further, the tubular structure 802 defines a first end 804 and a second end 806 opposite the first end 804. The second end 806 is disposed proximal to the wound dressing 110. The tubular structure 802 includes a first circular shape 803 at the first end 804 and a second circular shape 805 at the second end 806. Further, the tubular structure 802 includes a rectangular shape 807 extending between the first circular shape 803 and the second circular shape 805. The tubular structure 802 includes a first film 808 that is fluid impermeable. The first film 808 is coupled to the tubing 112 (see FIG. 1) for directing the negative pressure towards the wound site 12.

Referring now to FIG. 8C, the tubular structure 802 further includes a second film 810 connected to the first film 808. The second film 810 includes a wound port 812 disposed proximal to the second end 806 of the tubular structure 802 and fluidly communicating with the wound dressing 110. The first film 808 and the second film 810 together form an interior 814 of the tubular structure 802. The second film 810 is fluid impermeable.

The tubular structure 802 further includes a third film 816 at least partially disposed between and connected to each of the first film 808 and the second film 810. The third film 816 extends along the longitudinal axis A2 and separates the interior 814 into a first chamber 818 fluidly communicating with the therapy device 104 and a second chamber 820. The third film 816 is fluid impermeable and fluidly separates the first chamber 818 from the second chamber 820. Further, the wound port 812 fluidly communicates the second chamber 820 with the wound dressing 110. The first chamber 818 defines a first length L8-1 along the longitudinal axis A2 and the second chamber 820 defines a second length L8-2 along the longitudinal axis A2. In the illustrated embodiment of FIG. 8C, the second length L8-2 is equal to the first length L8-1. Alternatively, the second length L8-2 may be greater than the first length L8-1. Further, the third film 816 includes a plurality of filter openings 828 spaced apart from each other along the longitudinal axis A2. In some examples, the first film 808, the second film 810, and the third film 816 may be made from a transparent material. In such examples, the first film 808, the second film 810, and the third film 816 may provide a visual assessment when the tubular structure 802 is saturated with the wound exudate.

The first film 808 includes a plurality of first projections 822 extending into the first chamber 818 orthogonal to the longitudinal axis A2 and facing the third film 816. Each of the first projections 822 has a circular cross-section herein. Alternatively, each of the first projections 822 may have a square cross-section, a rectangular cross-section, an oval cross-section, a triangular cross-section, and the like. The first projections 822 provide a manifolding geometry to the first film 808 that may improve distribution of the negative pressure from the therapy device 104 within the first chamber 818. In some examples, the second film 810 may include a plurality of projections (not shown) extending into the second chamber 820 orthogonal to the longitudinal axis A2.

In the illustrated embodiment of FIG. 8C, the second film 810 includes a plurality of second projections 824 extending into the second chamber 820 orthogonal to the longitudinal axis A2. Each pf the second projections 824 has a circular cross-section herein. Alternatively, each of the second projections 824 may have a square cross-section, a rectangular cross-section, an oval cross-section, a triangular cross-section, and the like. The second projections 824 provide a manifolding geometry to the second film 810 that may improve distribution of the negative pressure from the therapy device 104 within the second chamber 820.

Further, the first film 808, the second film 810, and the third film 816 are similar in shape and size. Each of the first film 808, the second film 810, and the third film 816 may prevent fluid communication, including pneumatic communication, therethrough. Further, materials mentioned for each of the first, second, and third films 208, 210, 216 (see FIGS. 2A to 2C) may be applicable to each of the first film 808, the second film 810, and the third film 816.

Moreover, each of the first film 808, the second film 810, and the third film 816 may be connected to each other at each of the first end 804 and the second end 806. In an example, each of the first film 808, the second film 810, and the third film 816 may be connected to each other by a weld seam. In alternate examples, each of the first film 808, the second film 810, and the third film 816 may be connected to each other by bonding, stitching, an adhesive, a heat seal, and the like.

Further, the tubular structure 802 includes a plurality of filters 830 spaced apart from each other along the longitudinal axis A2. Each of the plurality of filters 830 is connected to the third film 816 and is configured to pneumatically communicate the first chamber 818 with the second chamber 820. Further, each of the plurality of filters 830 is offset from the wound port 812 with respect to the longitudinal axis A2. Furthermore, each of the plurality of filter openings 828 is configured to at least partially align with a corresponding filter 830 from the plurality of filters 830 therethrough. In some examples, a diameter of each filter 830 may be greater than a diameter of the filter openings 828, such that the filters 830 cover a corresponding filter opening 828. Further, when the filters 830 are connected to the third film 816, a portion of each filter 830 may overlap with the third film 816.

In the illustrated embodiment of FIG. 8C, the tubular structure 802 includes two filters 830. However, the tubular structure 802 may include more than two filters 830 based on, for example, dimensions of the tubular structure 802. In the illustrated embodiment of FIG. 8C, the filters 830 are disposed proximal to the first and second ends 804, 806, respectively. Further, the wound port 812 is located between the two filters 830 with respect to the longitudinal axis A2. In some examples, an additional filter may be positioned between the filters 830. Further, the filters 830 may be embodied as hydrophobic filters so that the filters 830 allow the negative pressure to flow therethrough, while preventing liquids, such as the wound exudate, from flowing therethrough. Thus, the third film 816 and the filters 830 may allow retention of the wound exudate within the second chamber 820 and prevent passage of any wound exudate to the first chamber 818 or towards the therapy device 104, thereby reducing a possibility of ingress of the wound exudate into the therapy device 104. Accordingly, the contamination of the components of the therapy device 104 (see FIG. 1), such as, the negative pressure source 106 (see FIG. 1) may be prevented. Further, the conduit device 800 may eliminate a need of hydrophobic filters between the conduit device 800 and the negative pressure source 106 for protecting the negative pressure source 106 from contamination due to contact with the wound exudate.

In some embodiments, the first film 808 includes a therapy port 832 disposed proximal to the first end 804 of the tubular structure 802 and fluidly communicating the first chamber 818 with the therapy device 104. In some embodiments, the tubular structure 802 includes a pad 834 connected to the first film 808 and covering the therapy port 832. The pad 834 is configured to provide fluid communication between the therapy device 104 and the therapy port 832. Further, the pad 834 may facilitate pneumatic communication between the negative pressure source 106 and the tubular structure 802. However, the pad 834 may prevent liquids to flow towards the therapy device 104. Accordingly, the pad 834 may be formed from a fluid impermeable material to prevent fluids from the tubular structure 802. In some examples, a material of the pad 834 may be same as the material of each of the first film 808, the second film 810, and the third film 816. Further, materials mentioned for the pad 234 (see FIGS. 2A to 2C) may be applicable to the pad 834.

The conduit device 800 includes an adhesive film 836 configured to removably connect the second film 810 with the wound dressing 110. The adhesive film 836 includes an annular shape herein and is concentric with the wound port 812. The adhesive film 836 may include a release liner which may be removed for removably connecting the second film 810 with the wound dressing 110. Further, materials mentioned for the adhesive film 236 (see FIGS. 2A to 2C) may be applicable to the adhesive film 836.

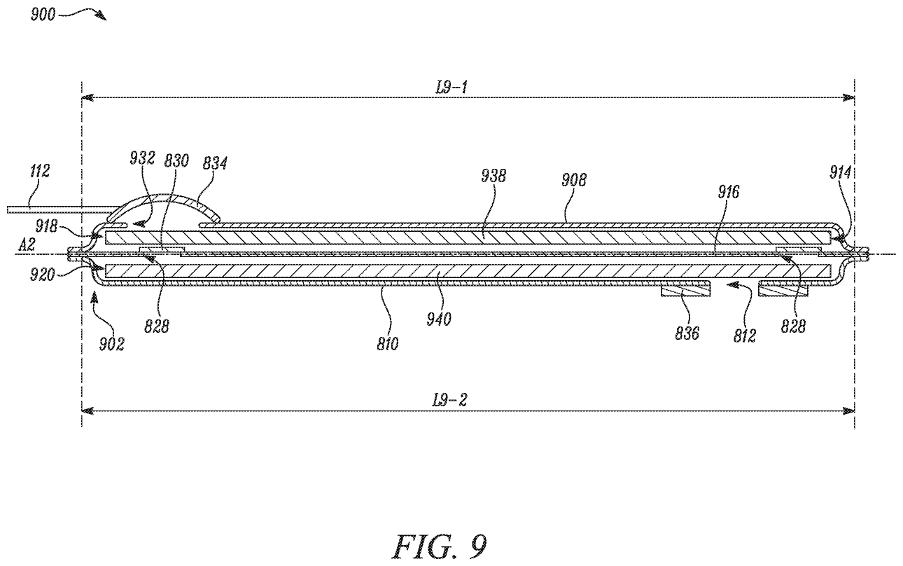

FIG. 9 is a schematic sectional side view of a conduit device 900 that may be associated with the NPWT system 100 of FIG. 1, according to an embodiment of the present disclosure. The conduit device 900 is functionally equivalent to the conduit device 800 shown in FIGS. 8A to 8C. Same parts are referred to herein by same numbers. As shown in FIG. 9, the conduit device 900 includes a tubular structure 902. The tubular structure 902 includes a first film 908 and a therapy port 932. The first film 908 is substantially similar to the first film 208 (see FIGS. 8A to 8C). However, the first film 908 does not include any projections. Further, the tubular structure 902 includes the second film 910 and the wound port 912. The second film 910 is substantially similar to the second film 810 (see FIGS. 8A to 8C). However, the second film 910 does not include any projections.

The tubular structure 902 further includes a third film 916. The third film 916 is substantially similar to the third film 816 (see FIGS. 8A to 8C). The third film 916 separates an interior 914 of the tubular structure 902 into a first chamber 918 fluidly communicating with the therapy device 104 (see FIG. 1) and a second chamber 920. The wound port 912 fluidly communicates the second chamber 920 with the wound dressing 110 (see FIG. 1). The first chamber 918 defines a first length L9-1 along the longitudinal axis A2 and the second chamber 920 defines a second length L9-2 along the longitudinal axis A2. In the illustrated embodiment of FIG. 9, the second length L9-2 is equal to the first length L9-1. Alternatively, the second length L9-2 may be greater than the first length L9-1.

Further, the tubular structure 902 includes the plurality of filters 830, the pad 834, and the adhesive film 836. Moreover, the tubular structure 902 includes a first manifold 938 disposed adjacent to the first film 908 within the first chamber 918. The first manifold 938 is positioned between the first film 908 and the third film 916. The first manifold 938 may be configured to allow passage of, or to channel, the negative pressure through the first chamber 918. In other words, the first manifold 938 may provide a flow passage across the first chamber 918. Further, the first film 908 and the first manifold 938 may be connected to each other so that the first manifold 938 may remain in position in the event that the conduit device 900 is moved, folded, or otherwise disturbed from the orientation illustrated in FIG. 9. In an example, the first film 908 may be connected to the first manifold 938 by a weld seam, bonding, stitching, an adhesive, a heat seal, and the like.

The tubular structure 902 further includes a second manifold 940 disposed adjacent to the second film 910 within the second chamber 920. The second manifold 940 is positioned between the second film 910 and the third film 916. The second manifold 940 may be configured to allow passage of, or to channel, the negative pressure as well as the wound exudate through the second chamber 920. In other words, the second manifold 940 may provide a flow passage across the second chamber 920. Further, in one example, the second manifold 940 may be connected to the second film 910 so that the second manifold 940 may remain in position in the event that the conduit device 900 is moved, folded, or otherwise disturbed from the orientation illustrated in FIG. 9. In an example, the second manifold 940 may be connected to the second film 910 by a weld seam, bonding, stitching, an adhesive, a heat seal, and the like. Alternatively, the first manifold 938, the second manifold 940, the first film 908, the second film 910, and the third film 916 may be connected to each other at either ends of the tubular structure 902 by a weld seam, bonding, stitching, an adhesive, a heat seal, and the like. In some embodiments, a material of each of the first and second manifolds 938, 940 may be similar to materials of the first and second manifolds 338, 340 (see FIG. 3).

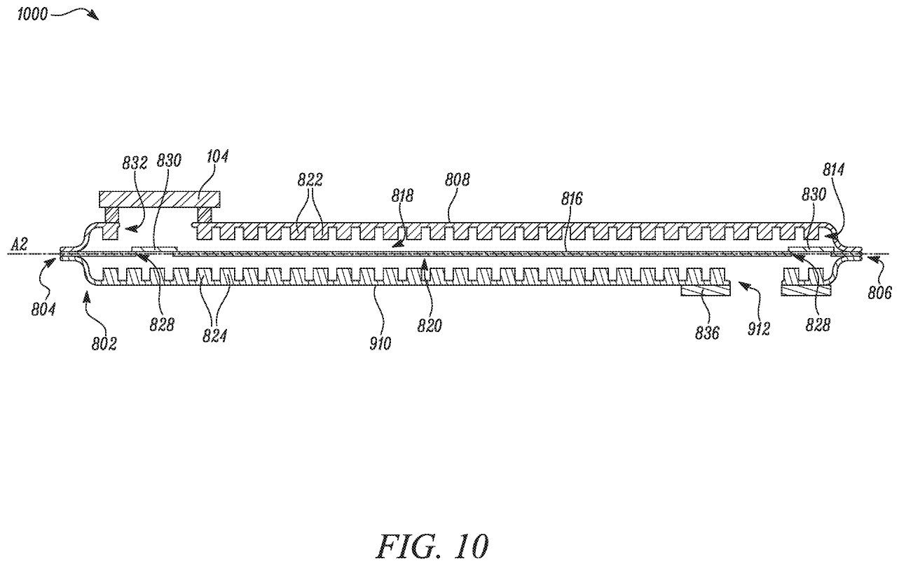

FIG. 10 is a schematic sectional side view of a conduit device 1000 that may be associated with the NPWT system 100 of FIG. 1, according to another embodiment of the present disclosure. The conduit device 1000 is functionally equivalent to the conduit device 800 shown in FIGS. 8A to 8C. Same parts are referred to herein by same numbers. As shown in FIG. 10, the conduit device 1000 includes the tubular structure 802, the first film 808, the therapy port 832. The first film 808 includes the first projections 822. The first projections 822 extend between the therapy port 832 and the second end 806. Further, the tubular structure 802 includes the second film 810, the third film 816, the wound port 812, the interior 814, the first chamber 818, and the second chamber 820. Furthermore, the second film 810 includes the second projections 824. The second projections 824 extend between the first end 804 and the second end 806.

The tubular structure 802 includes the plurality of filters 830 and the adhesive film 836. Further, in the illustrated embodiment of FIG. 10, the therapy device 104 is integral with the tubular structure 802 and connected to the first film 808. Specifically, the therapy device 104 is coupled to the therapy port 832 and is in direct fluid communication with the therapy port 832. The conduit device 1000 may eliminate a step of coupling the therapy device 104 with the tubular structure 802, thereby allowing quicker assembly of the NPWT system 100. Further, the conduit device 1000 may also eliminate a requirement of the tubing 112 (see FIG. 1) and the connector 114 (see FIG. 1).

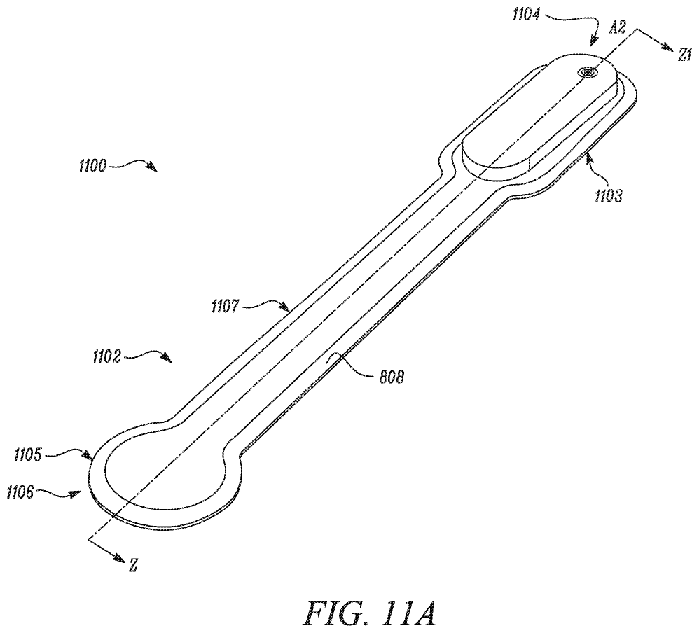

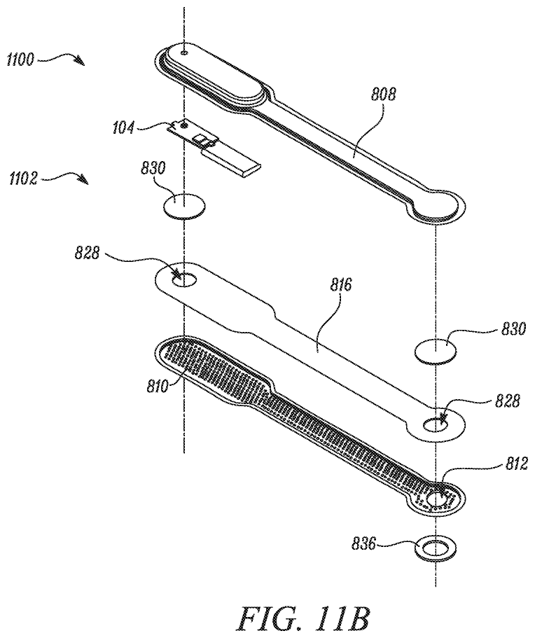

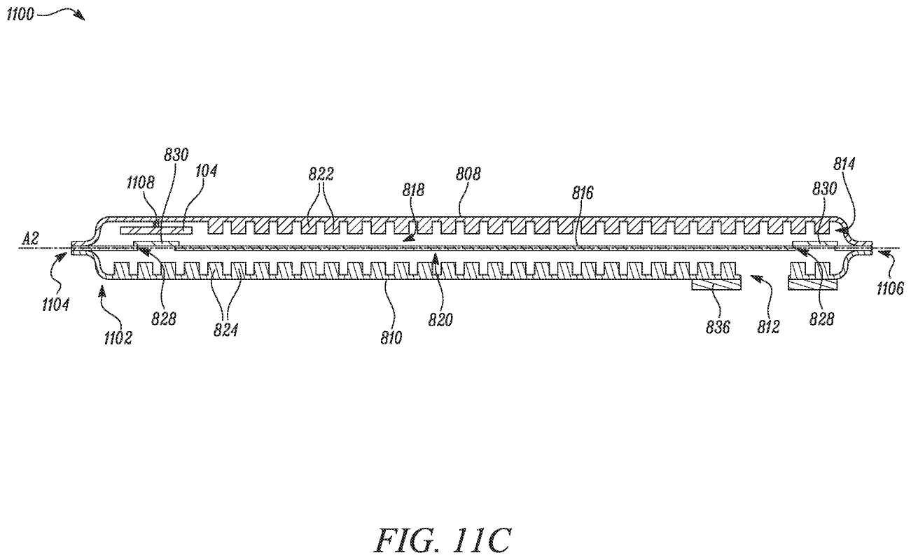

FIGS. 11A, 11B, and 11C illustrate a conduit device 1100 that may be associated with the NPWT system 100 of FIG. 1, according to yet another embodiment of the present disclosure. The conduit device 1100 is functionally equivalent to the conduit device 800 shown in FIGS. 8A to 8C. Same parts are referred to herein by same numbers. As shown in FIG. 11A, the conduit device 1100 includes the tubular structure 1102. The tubular structure 1102 includes a first shape 1103 at a first end 1104 and a second shape 1105 at a second end 1106. The first shape 1103 includes a capsule shape and the second shape 1105 includes a circular shape. Further, the tubular structure 1102 includes a third shape 1107 extending between the first shape 1103 and the second shape 1105. The third shape 1107 includes a rectangular shape.

As shown in FIG. 11B, the tubular structure 1102 includes the first film 808, the second film 810, the third film 816, the wound port 812, the interior 814, the first chamber 818, and the second chamber 820. As shown in FIG. 11C, the first film 808 includes the first projections 822. Further, the second film 810 includes the second projections 824. The second projections 824 extend between the first end 1104 and the second end 1106.

Further, the tubular structure 1102 includes the plurality of filters 830 and the adhesive film 836. In the illustrated embodiment of FIG. 11C, the therapy device 104 is disposed within the first chamber 818 and adjacent to the first film 808. Specifically, the therapy device 104 is in direct fluid communication with the first chamber 818. Further, the therapy device 104 is disposed proximate to and in partial alignment with the filter 830 disposed proximate to the first end 1104. In an example, a gasket 1108 may connect the therapy device 104 to the first film 808. Alternatively, the gasket 1108 may connect the therapy device 104 to the third film 816. In other examples, the therapy device 104 may be connected to the first film 808 or the third film 816 by bonding, an adhesive, and the like. The conduit device 1100 may eliminate a step of coupling the therapy device 104 with the tubular structure 1102, thereby allowing quicker assembly of the NPWT system 100. Further, the conduit device 1100 may also eliminate a requirement of the tubing 112 (see FIG. 1) and the connector 114 (see FIG. 1).

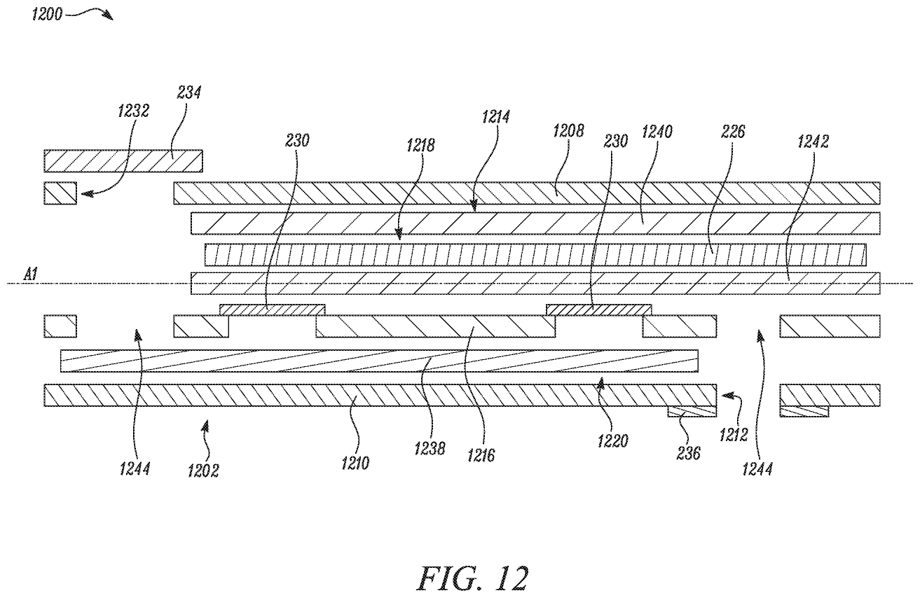

FIG. 12 is a schematic sectional side view of a conduit device 1200 that may be associated with the NPWT system 100 of FIG. 1, according to an embodiment of the present disclosure. The conduit device 1200 is functionally equivalent to the conduit device 200 shown in FIGS. 1 to 2C. Same parts are referred to herein by same numbers. As shown in FIG. 12, the conduit device 1200 includes a tubular structure 1202. The tubular structure 1202 includes a first film 1208 and a therapy port 1232 that may be substantially similar to the first film 208 and the therapy port 232 (see FIGS. 2A to 2C). The first film 1208 may include a high MVTR film. The first film 1208 does not include any projections. Further, the tubular structure 1202 includes a second film 1210 and a wound port 1212. The second film 1210 and the wound port 1212 may be substantially similar to the second film 210 and the wound port 212 (see FIGS. 2A to 2C).

The tubular structure 1202 further includes a third film 1216. The third film 1216 may be substantially similar to the third film 216 (see FIGS. 2A to 2C). The third film 1216 does not include any projections. The third film 1216 separates an interior 1214 of the tubular structure 1202 into a first chamber 1218 fluidly communicating with the therapy device 104 (see FIG. 1) and a second chamber 1220. The wound port 1212 fluidly communicates the second chamber 1220 with the wound dressing 110 (see FIG. 1). Further, the third film 1216 defines a pair of openings 1244 that are spaced apart from each other along the longitudinal axis A1. Each opening 1244 fluidly communicates the first chamber 1208 with the second chamber 1210 so that the wound exudate received from the wound port 1212 may be directed towards the absorbent 226.

Further, the tubular structure 1202 includes the plurality of filters 230, the absorbent 226, the pad 234, and the adhesive film 236. However, in the illustrated embodiment of FIG. 12, the absorbent 226 is disposed in the first chamber 1218 and offset from the wound port 1212 with respect to the longitudinal axis A1. Moreover, the tubular structure 1202 includes a first manifold 1238 disposed adjacent to the second film 1210 within the second chamber 1220. The first manifold 1238 may be configured to allow passage of, or to channel, the negative pressure as well as the wound exudate through the second chamber 1220. In other words, the first manifold 1238 may provide a flow passage across the second chamber 1220. Further, the second film 1210 and the first manifold 1238 may be connected to each other so that the first manifold 1238 may remain in position in the event that the conduit device 1200 is moved, folded, or otherwise disturbed from the orientation illustrated in FIG. 12. In an example, the second chamber 1220 and the first manifold 1238 may be connected to each other by a weld seam, bonding, stitching, an adhesive, a heat seal, and the like.

The tubular structure 1202 further includes a second manifold 1240 disposed adjacent to the first film 1208 within the first chamber 1218. The second manifold 1240 is positioned between the absorbent 226 and the first film 1208. The second manifold 1240 may be configured to allow passage of, or to channel, the negative pressure through the first chamber 1218. In other words, the second manifold 1240 may provide a flow passage across the first chamber 1218. Further, in one example, the second manifold 1240 may be connected to the first film 1208 or the absorbent 226 so that the second manifold 1240 may remain in position in the event that the conduit device 1200 is moved, folded, or otherwise disturbed from the orientation illustrated in FIG. 12. In an example, the second manifold 1240 may be connected to the first film 1208 or the absorbent 226 by a weld seam, bonding, stitching, an adhesive, a heat seal, and the like.

Further, the tubular structure 1202 includes a third manifold 1242 disposed within the first chamber 1218. The third manifold 1242 is positioned between the third film 1216 and the absorbent 226. The third manifold 1242 may be configured to allow passage of, or to channel, the negative pressure as well as the wound exudate through the first chamber 1218. In other words, the third manifold 1242 may provide a flow passage across the first chamber 1218. Further, in one example, the third manifold 1242 may be connected to the third film 1216 or the absorbent 226 so that the third manifold 1242 may remain in position in the event that the conduit device 1200 is moved, folded, or otherwise disturbed from the orientation illustrated in FIG. 12. In an example, the third manifold 1242 may be connected to the third film 1216 or the absorbent 226 by a weld seam, bonding, stitching, an adhesive, a heat seal, and the like. Alternatively, the first manifold 1238, the second manifold 1240, the third manifold 1242, the first film 1208, the second film 1210, and the third film 1216 may be connected to each other by a weld seam, bonding, stitching, an adhesive, a heat seal, and the like. Further, materials mentioned for each of the first and second manifolds 338, 340 (see FIG. 3) may be applicable to each of the first, second, and third manifolds 1238, 1240, 1242.

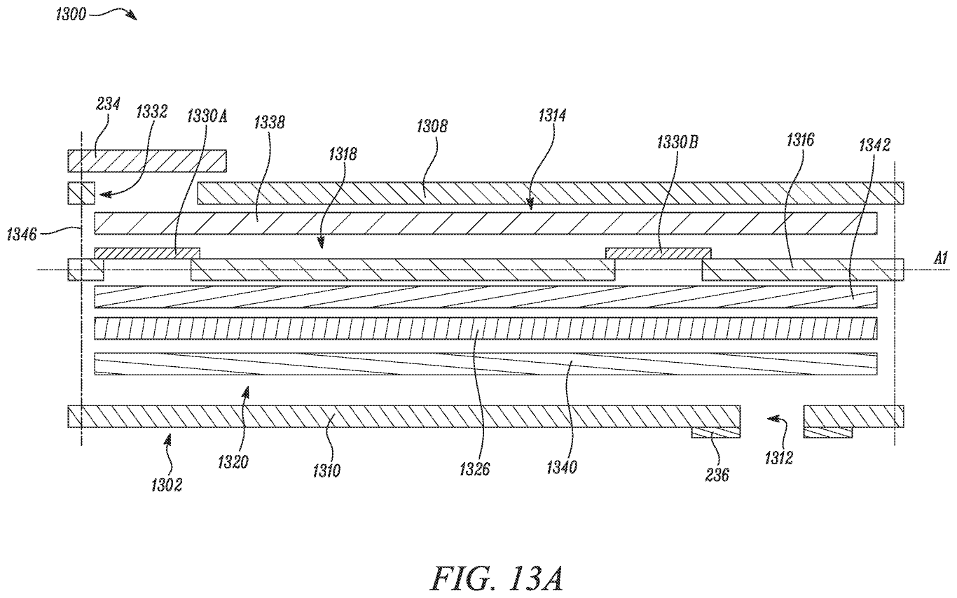

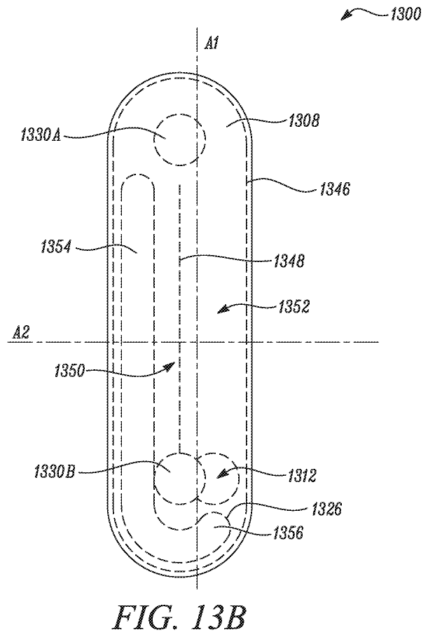

FIG. 13A is a schematic sectional side view of a conduit device 1300 that may be associated with the NPWT system 100 of FIG. 1, according to another embodiment of the present disclosure. The conduit device 1300 is functionally equivalent to the conduit device 200 shown in FIGS. 1 to 2C. Same parts are referred to herein by same numbers. As shown in FIG. 13A, the conduit device 1300 includes a tubular structure 1302. The tubular structure 1302 includes a first film 1308 and a therapy port 1332. The first film 1308 and a therapy port 1332 is substantially similar to the first film 208 and the therapy port 232 (see FIGS. 2A to 2C). The first film 1308 does not include any projections. Further, the tubular structure 1302 includes a second film 1310 and a wound port 1312. The second film 1310 and the wound port 1312 may be substantially similar to the second film 210 and the wound port 212 (see FIGS. 2A to 2C).

The tubular structure 1302 further includes a third film 1316. The third film 1316 is substantially similar to the third film 216 (see FIGS. 2A to 2C). The third film 1316 does not include any projections. The third film 1316 separates an interior 1314 of the tubular structure 1302 into a first chamber 1318 fluidly communicating with the therapy device 104 (see FIG. 1) and a second chamber 1320. The wound port 1312 fluidly communicates the second chamber 1320 with the wound dressing 110 (see FIG. 1).

Further, the tubular structure 1302 includes a plurality of filters 1330A, 1330B, an absorbent 1326, the pad 234, and the adhesive film 236. The absorbent 1326 is substantially similar in material to the absorbent 226 as explained in FIG. 2C. The absorbent 1326 is disposed in the second chamber 1320 and offset from the wound port 1312 with respect to the lateral axis A2 (see FIG. 13B). Further, at least one of the plurality of filters 1330A, 1330B is offset from the wound port 1312 with respect to the lateral axis A2. In the illustrated embodiment of FIG. 12B, each filter 1330A, 1330B is offset from the wound port 1312 with respect to the lateral axis A2. Further, the filter 1330A is also offset from the wound port 1312 with respect to the longitudinal axis A1.

Moreover, the tubular structure 1302 includes a first manifold 1338 disposed adjacent to the first film 1308 within the first chamber 1318. The first manifold 1338 may be configured to allow passage of, or to channel, the negative pressure through the first chamber 1318. In other words, the first manifold 1338 may provide a flow passage across the first chamber 1318. Further, the first film 1308 and the first manifold 1338 may be connected to each other so that the first manifold 1338 may remain in position in the event that the conduit device 1300 is moved, folded, or otherwise disturbed from the orientation illustrated in FIG. 13A. In an example, the first film 1308 and the first manifold 1338 may be connected to each other by a weld seam, bonding, stitching, an adhesive, a heat seal, and the like.

The tubular structure 1302 further includes a second manifold 1340 disposed adjacent to the second film 1310 within the second chamber 1320. The second manifold 1340 is positioned between the absorbent 1326 and the second film 1310. The second manifold 1340 may be configured to allow passage of, or to channel, the negative pressure as well as the wound exudate through the second chamber 1320. In other words, the second manifold 1340 may provide a flow passage across the second chamber 1320. Further, in one example, the second manifold 1340 may be connected to the second film 1310 or the absorbent 1326 so that the second manifold 1340 may remain in position in the event that the conduit device 1300 is moved, folded, or otherwise disturbed from the orientation illustrated in FIG. 13A. In an example, the second manifold 1340 may be connected to the second film 1310 or the absorbent 1326 by a weld seam, bonding, stitching, an adhesive, a heat seal, and the like.

Further, the tubular structure 1302 includes a third manifold 1342 disposed within the second chamber 1320. The third manifold 1342 is positioned between the third film 1316 and the absorbent 1326. The third manifold 1342 may be configured to allow passage of, or to channel, the negative pressure as well as the wound exudate through the second chamber 1320. In other words, the third manifold 1342 may provide a flow passage across the second chamber 1320. Further, in one example, the third manifold 1342 may be connected to the third film 1316 or the absorbent 1326 so that the third manifold 1342 may remain in position in the event that the conduit device 1300 is moved, folded, or otherwise disturbed from the orientation illustrated in FIG. 13A. In an example, the third manifold 1342 may be connected to the third film 1316 or the absorbent 1326 by a weld seam, bonding, stitching, an adhesive, a heat seal, and the like. Alternatively, the first manifold 1338, the second manifold 1340, the third manifold 1342, the first film 1308, the second film 1310, and the third film 1316 may be connected to each other by a weld seam, bonding, stitching, an adhesive, a heat seal, and the like. Further, materials mentioned for each of the first and second manifolds 338, 340 (see FIG. 3) may be applicable to each of the first, second, and third manifolds 1338, 1340, 1342.

Referring now to FIG. 13B, each of the first film 1308, the second film 1310, and the third film 1316 are connected to each other at their corresponding peripheries by a first weld seam 1346. Further, the second film 1310 is connected to the third film 1316 by a second weld seam 1348. The second weld seam 1348 at least partially divides the second chamber 1320 into a first sub-chamber 1350 and a second sub-chamber 1352. Further, the wound port 1312 is disposed in the second sub-chamber 1352. The location of the wound port 1312 allows the wound exudate to enter and be stored in the absorbent 1326. Moreover, a free path as defined by the second sub-chamber 1352 may be used to apply the negative pressure to the wound site 12 (see FIG. 1) even when the absorbent 1326 is saturated with the wound exudate. Further, the filter 1330B is disposed centrally between the first and second sub-chambers 1350, 1352 defined by the second chamber 1320 so that the negative pressure as well as the wound exudate may be equally distributed across the first and second sub-chambers 1350, 1352.

Further, as shown in FIG. 13B, the absorbent 1326 is offset from the weld seam 1348 along the lateral axis A2. Furthermore, the absorbent 1326 is offset from each of the plurality of filters 1330A, 1330B with respect to the longitudinal axis A1. Moreover, the absorbent 1326 has a straight portion 1354 and a curved portion 1356 that at least partially surrounds the filter 1330B. The straight portion 1354 is disposed within the first sub-chamber 1350. The location and design of the absorbent 1326 as described herein may allow uniform pressure distribution in the second chamber 1320.

Referring to FIGS. 1 to 13B, the conduit device 200, 300, 400, 500, 600, 700, 800, 900, 1000, 1100, 1200, 1300 provides an effective way of managing low levels of the wound exudate that is removed from the wound site 12. In one particular embodiment, the conduit device 200, 300, 400, 500, 600, 700, 800, 900, 1000, 1100, 1200, 1300 may provide a multi-directional flexible canister which can be used in a range of different form factors or orientations as a multi-point pressure manifold solution. Further, multiple orientation use of the conduit device 200, 300, 400, 500, 600, 700, 800, 900, 1000, 1100, 1200, 1300 may permit use of the conduit device 200, 300, 400, 500, 600, 700, 800, 900, 1000, 1100, 1200, 1300 in a wider variety of locations, and persons. In addition, multiple orientation use of the conduit device 200, 300, 400, 500, 600, 700, 800, 900, 1000, 1100, 1200, 1300 may permit the conduit device 200, 300, 400, 500, 600, 700, 800, 900, 1000, 1100, 1200, 1300 to be used in a mobile environment. Therefore, the conduit device 200, 300, 400, 500, 600, 700, 800, 900, 1000, 1100, 1200, 1300 may eliminate the requirement of a separate rigid canister to store the wound exudate thereby reducing a weight of the NPWT system 100, while improving a portability of the NPWT system 100.

Overall, the conduit device 200, 300, 400, 500, 600, 700, 800, 900, 1000, 1100, 1200, 1300 described herein may be usable in multiple orientations, may be simple to use, may improve patient comfort, may reduce a possibility of infection at the wound site 12 by directing the wound exudate away from the wound site 12, may improve an efficacy and the portability of the NPWT system 100, and may effectively manage wound exudate removed from the wound site 12.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below, or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example.

Various examples have been described. These and other examples are within the scope of the following claims.

1. A conduit device for use with a negative pressure wound therapy system, the conduit device comprising:
a tubular structure configured to fluidly connect to each of a therapy device and a wound dressing located at a wound site, the tubular structure extending along a longitudinal axis and a lateral axis orthogonal to the longitudinal axis, the tubular structure defining a first end and a second end opposite the first end, wherein the second end is disposed proximal to the wound dressing, the tubular structure comprising:
a first film that is fluid impermeable;
a second film connected to the first film, wherein the second film comprising a wound port disposed proximal to the second end of the tubular structure and fluidly communicating with the wound dressing, wherein the first film and the second film together form an interior of the tubular structure, and wherein the second film is fluid impermeable;
a third film at least partially disposed between and connected to each of the first film and the second film, wherein the third film extends along the longitudinal axis and separates the interior into a first chamber fluidly communicating with the therapy device and a second chamber, wherein the third film is fluid impermeable and fluidly separates the first chamber from the second chamber, and wherein the wound port fluidly communicates the second chamber with the wound dressing;
a plurality of filters spaced apart from each other along the longitudinal axis, wherein each of the plurality of filters is connected to the third film and is configured to pneumatically communicate the first chamber with the second chamber, and wherein each of the plurality of filters is offset from the wound port with respect to at least one of the longitudinal axis and the lateral axis; and
an absorbent disposed in the first chamber or the second chamber and offset from the wound port with respect to at least one of the longitudinal axis and the lateral axis.

2. The conduit device of claim 1, wherein the plurality of filters are disposed along a first linear length of the third film along the longitudinal axis, wherein the absorbent defines a second linear length along the longitudinal axis, and wherein the second linear length is greater than the first linear length.

3. The conduit device of claim 1, wherein the plurality of filters are disposed along a first linear length of the third film along the longitudinal axis, wherein the absorbent defines a second linear length along the longitudinal axis, and wherein the second linear length is less than the first linear length.

4. The conduit device of claim 1, wherein:
the first film comprises a therapy port disposed proximal to the first end of the tubular structure and fluidly communicating the first chamber with the therapy device; and
the conduit device further comprises a pad connected to the first film and covering the therapy port, wherein the pad is configured to provide fluid communication between the therapy device and the therapy port.

5. (canceled)

6. The conduit device of claim 1, wherein the third film comprises a plurality of filter openings spaced apart from each other along the longitudinal axis, and wherein each of the plurality of filter openings is configured to at least partially align with a corresponding filter from the plurality of filters therethrough.

7. The conduit device of claim 1, further comprising an adhesive film configured to removably connect the second film with the wound dressing.

8. The conduit device of claim 1, wherein the first chamber defines a first length along the longitudinal axis and the second chamber defines a second length along the longitudinal axis, and wherein the second length is greater than the first length.

9. The conduit device of claim 1, wherein the first chamber defines a first length along the longitudinal axis and the second chamber defines a second length along the longitudinal axis, and wherein the first length is equal to the second length.

10. The conduit device of claim 1, wherein the first film comprises a plurality of first projections extending into the first chamber orthogonal to the longitudinal axis and facing the third film, and wherein the third film comprises a plurality of second projections extending into the second chamber orthogonal to the longitudinal axis and configured to at least partially engage with the absorbent.

11. The conduit device of claim 1, wherein the absorbent is directly adjacent to each of the second film and the third film.

12. The conduit device of claim 1, wherein the absorbent is disposed in the second chamber and offset from the wound port with respect to the longitudinal axis, and wherein the tubular structure further comprises:
a first manifold disposed adjacent to the first film within the first chamber; and
a second manifold disposed adjacent to the second film within the second chamber, wherein the second manifold is positioned between the absorbent and the second film.

13. (canceled)

14. The conduit device of claim 1, wherein the absorbent is disposed in the second chamber and offset from the wound port with respect to the longitudinal axis, and wherein the tubular structure further comprises:
a first manifold disposed adjacent to the first film within the first chamber;
a second manifold disposed adjacent to the second film within the second chamber; and
a third manifold disposed within the second chamber, wherein the third manifold is disposed adjacent to the absorbent;
a fourth film connected to each of the first film and the second film proximal to the first end of the tubular structure, wherein the fourth film extends along the longitudinal axis and comprises a free end spaced apart from the second film proximal to the second end of the tubular structure, such that a fluid passage is defined between the fourth film and the second film within the second chamber, wherein the fourth film is disposed between the second manifold and the third manifold, and wherein the fourth film is fluid impermeable;
at least one auxiliary filter connected to the fourth film, wherein the at least one auxiliary filter is connected to the fourth film and is configured to provide pneumatic communication therethrough; and
a fourth manifold disposed within the second chamber, wherein the fourth manifold is positioned between the third film and the absorbent.

15. The conduit device of claim 1, wherein the absorbent is disposed in the first chamber and offset from the wound port with respect to the longitudinal axis, and wherein the tubular structure further comprises:
a first manifold disposed adjacent to the second film within the second chamber;
a second manifold disposed adjacent to the first film within the first chamber, wherein the second manifold is positioned between the absorbent and the first film; and
a third manifold disposed within the first chamber, wherein the third manifold is positioned between the third film and the absorbent.

16. The conduit device of claim 1, wherein the absorbent is disposed in the second chamber and offset from the wound port with respect to the lateral axis, and wherein at least one of the plurality of filters is offset from the wound port with respect to the lateral axis.

17. The conduit device of claim 1, wherein the absorbent is at least partially aligned with at least one of the plurality of filters with respect to the longitudinal axis.

18. The conduit device of claim 1, wherein the absorbent is offset from each of the plurality of filters with respect to the longitudinal axis.

19.-21. (canceled)

22. A conduit device for use with a negative pressure wound therapy system, the conduit device comprising:
a tubular structure configured to fluidly connect to each of a therapy device and a wound dressing located at a wound site, the tubular structure extending along a longitudinal axis, the tubular structure defining a first end and a second end opposite the first end, wherein the second end is disposed proximal to the wound dressing, the tubular structure comprising:
a first film that is fluid impermeable;
a second film connected to the first film, wherein the second film comprises a wound port disposed proximal to the second end of the tubular structure and fluidly communicating with the wound dressing, wherein the first film and the second film together form an interior of the tubular structure, and wherein the second film is fluid impermeable;
a third film at least partially disposed between and connected to each of the first film and the second film, wherein the third film extends along the longitudinal axis and separates the interior into a first chamber fluidly communicating with the therapy device and a second chamber, wherein the third film is fluid impermeable and fluidly separates the first chamber from the second chamber, wherein the wound port fluidly communicates the second chamber with the wound dressing, wherein the first chamber defines a first length along the longitudinal axis and the second chamber defines a second length along the longitudinal axis, and wherein the first length is equal to the second length; and
a plurality of filters spaced apart from each other along the longitudinal axis, wherein each of the plurality of filters is connected to the third film and is configured to pneumatically communicate the first chamber with the second chamber, and wherein each of the plurality of filters is offset from the wound port with respect to the longitudinal axis.

23. The conduit device of claim 22, wherein the first film comprises a therapy port disposed proximal to the first end of the tubular structure and fluidly communicating the first chamber with the therapy device.

24. The conduit device of claim 23, further comprising a pad connected to the first film and covering the therapy port, and wherein the pad is configured to provide fluid communication between the therapy device and the therapy port.

25. The conduit device of claim 22, wherein the third film comprises a plurality of filter openings spaced apart from each other along the longitudinal axis, and wherein each of the plurality of filter openings is configured to at least partially align with a corresponding filter from the plurality of filters therethrough.

26.-31. (canceled)
